SEQUENCE LISTING (90 sequences)

SEQ ID NO: 1 - DNA, 21 bp, Artificial Sequence
  Primer
acttcagattcccccacaaca

SEQ ID NO: 2 - DNA, 21 bp, Artificial Sequence
  Primer
tgcttccttcacgacattcaa

SEQ ID NO: 3 - DNA, 19 bp, Artificial Sequence
  Primer
atgctgagtgcggcagaag

SEQ ID NO: 4 - DNA, 24 bp, Artificial Sequence
  Primer
atgctgactgtagcagaaggaatc

SEQ ID NO: 5 - DNA, 23 bp, Artificial Sequence
  Primer
atgctgaattcagcagaaggcat

SEQ ID NO: 6 - DNA, 26 bp, Artificial Sequence
  Primer
atgctctgggaactgtatgtatttgt

SEQ ID NO: 7 - DNA, 20 bp, Artificial Sequence
  Primer
atggtggcagttctacagag

SEQ ID NO: 8 - DNA, 19 bp, Artificial Sequence
  Primer
atgaagcacttttggaaga

SEQ ID NO: 9 - DNA, 22 bp, Artificial Sequence
  Primer
atggaaggtcatatgctcttct

SEQ ID NO: 10 - DNA, 27 bp, Artificial Sequence
  Primer
atgatgggtattgccatagatatctta

SEQ ID NO: 11 - DNA, 25 bp, Artificial Sequence
  Primer
atgctaccaacattatcagttttct

SEQ ID NO: 12 - DNA, 33 bp, Artificial Sequence
  Primer
ctaaaagaactttaatccttgcagtacctttac

SEQ ID NO: 13 - DNA, 24 bp, Artificial Sequence
  Primer
ctaccatgtcactctgacgtcctt

SEQ ID NO: 14 - DNA, 30 bp, Artificial Sequence
  Primer
ttatttgactctgaaatttccttttgtctc

SEQ ID NO: 15 - DNA, 35 bp, Artificial Sequence
  Primer
ctacttgtagaaacagaaaatcttctttgctttag

SEQ ID NO: 16 - DNA, 33 bp, Artificial Sequence
  Primer
ctatacttctatatttttaaacctgtaccagag

SEQ ID NO: 17 - DNA, 28 bp, Artificial Sequence
  Primer
ctagaatatacaacataatctaaagtac

SEQ ID NO: 18 - DNA, 24 bp, Artificial Sequence
  Primer
ttaagatggcattatacaggcttc

SEQ ID NO: 19 - DNA, 20 bp, Artificial Sequence
  Primer
tcagggaaccaacatccgta

SEQ ID NO: 20 - DNA, 18 bp, Artificial Sequence
  Primer
ctaggccacccagaatcc

SEQ ID NO: 21 - DNA, 23 bp, Artificial Sequence
  Primer
ctcctgctaatcttctctttgtg

SEQ ID NO: 22 - DNA, 24 bp, Artificial Sequence
  Primer
gggttcttggtatcattattggac

SEQ ID NO: 23 - DNA, 19 bp, Artificial Sequence
  Primer
gcaacacatcctggctgat

SEQ ID NO: 24 - DNA, 20 bp, Artificial Sequence
  Primer
aaggcatcctcctttccatt

SEQ ID NO: 25 - DNA, 20 bp, Artificial Sequence
  Primer
agccacattcttctcaacct

SEQ ID NO: 26 - DNA, 20 bp, Artificial Sequence
  Primer
ggcatcctcctttgtgttgt

SEQ ID NO: 27 - DNA, 22 bp, Artificial Sequence
  Primer
gtttctcctgttgaaacggact

SEQ ID NO: 28 - DNA, 24 bp, Artificial Sequence
  Primer
gtcaaattcaggtgttaggaagtc

SEQ ID NO: 29 - DNA, 23 bp, Artificial Sequence
  Primer
ctttctcatgctcatcttctcac

SEQ ID NO: 30 - DNA, 21 bp, Artificial Sequence
  Primer
ccacggtaatgttttctttgc

SEQ ID NO: 31 - DNA, 19 bp, Artificial Sequence
  Primer
cggctgccactcacttatc

SEQ ID NO: 32 - DNA, 24 bp, Artificial Sequence
  Primer
cctttggtgtatccttgatagctt

SEQ ID NO: 33 - DNA, 20 bp, Artificial Sequence
  Primer
aaggtttggagtgctctgct

SEQ ID NO: 34 - DNA, 20 bp, Artificial Sequence
  Primer
ccctgtggacacatcacaag

SEQ ID NO: 35 - DNA, 20 bp, Artificial Sequence
  Primer
cactgggtgcagatgaaaca

SEQ ID NO: 36 - DNA, 22 bp, Artificial Sequence
  Primer
aaggaacccaagactcagtgac

SEQ ID NO: 37 - DNA, 21 bp, Artificial Sequence
  Primer
tgttaacgaactggcattcac

SEQ ID NO: 38 - DNA, 24 bp, Artificial Sequence
  Primer
ctggtcttattggagatgattgtg

SEQ ID NO: 39 - DNA, 22 bp, Artificial Sequence
  Primer
tcttctctttatggagccacct

SEQ ID NO: 40 - DNA, 22 bp, Artificial Sequence
  Primer
cattaaagccttgcaaactgtg

SEQ ID NO: 41 - DNA, 23 bp, Artificial Sequence
  Primer
ctccaccatcatactaattgcag

SEQ ID NO: 42 - DNA, 20 bp, Artificial Sequence
  Primer
aaggccttgcacatggtagt

SEQ ID NO: 43 - DNA, 20 bp, Artificial Sequence
  Primer
gtgtgtgggattggtcaaca

SEQ ID NO: 44 - DNA, 22 bp, Artificial Sequence
  Primer
caaagatgcagagatgtccttg

SEQ ID NO: 45 - DNA, 21 bp, Artificial Sequence
  Primer
tgcattcattgcactggtaaa

SEQ ID NO: 46 - DNA, 19 bp, Artificial Sequence
  Primer
tagcccacatttcccatcc

SEQ ID NO: 47 - DNA, 20 bp, Artificial Sequence
  Primer
gcctgggaagtggtaaccta

SEQ ID NO: 48 - DNA, 20 bp, Artificial Sequence
  Primer
ccatcatgtccacaggagaa

SEQ ID NO: 49 - DNA, 21 bp, Artificial Sequence
  Primer
ggacaatgaggctttatggaa

SEQ ID NO: 50 - DNA, 20 bp, Artificial Sequence
  Primer
ctggctcaaatggagagctt

SEQ ID NO: 51 - DNA, 20 bp, Artificial Sequence
  Primer
caaaccaagtgagcctctgg

SEQ ID NO: 52 - DNA, 22 bp, Artificial Sequence
  Primer
agtgcaccataggtatcattgc

SEQ ID NO: 53 - DNA, 21 bp, Artificial Sequence
  Primer
gaagaacatgcaacacaatgc

SEQ ID NO: 54 - DNA, 20 bp, Artificial Sequence
  Primer
cattggcctctatgttgcag

SEQ ID NO: 55 - DNA, 22 bp, Artificial Sequence
  Primer
aagcagaaaatcatagggctga

SEQ ID NO: 56 - DNA, 19 bp, Artificial Sequence
  Primer
gggtctctgtgtcttctgg

SEQ ID NO: 57 - DNA, 23 bp, Artificial Sequence
  Primer
accatccaggaaatagtaaggag

SEQ ID NO: 58 - DNA, 20 bp, Artificial Sequence
  Primer
ccccatattggcaaaaacat

SEQ ID NO: 59 - DNA, 24 bp, Artificial Sequence
  Primer
gtgcaataaatgtgttccctaaaa

SEQ ID NO: 60 - DNA, 21 bp, Artificial Sequence
  Primer
agcatgtaatgatagccacca

SEQ ID NO: 61 - DNA, 24 bp, Artificial Sequence
  Primer
tgcaatatatgtgtcccctaaaac

SEQ ID NO: 62 - DNA, 21 bp, Artificial Sequence
  Primer
gtgagggctgaaatcagaaga

SEQ ID NO: 63 - DNA, 20 bp, Artificial Sequence
  Primer
cacagggagaagatgagcag

SEQ ID NO: 64 - DNA, 20 bp, Artificial Sequence
  Primer
ggcatctctaggtggtttgg

SEQ ID NO: 65 - DNA, 20 bp, Artificial Sequence
  Primer
tggtgctgatgtctctgcat

SEQ ID NO: 66 - DNA, 27 bp, Artificial Sequence
  Primer
cagcactttaatagttgcagtatcatt

SEQ ID NO: 67 - DNA, 23 bp, Artificial Sequence
  Primer
ctgcatcttccttacatgtttca

SEQ ID NO: 68 - DNA, 21 bp, Artificial Sequence
  Primer
agctgttcttgcaacctgtgt

SEQ ID NO: 69 - DNA, 22 bp, Artificial Sequence
  Primer
tcacagtttgtagggctttgaa

SEQ ID NO: 70 - DNA, 24 bp, Artificial Sequence
  Primer
cttcagaacagtgaactgagcttt

SEQ ID NO: 71 - DNA, 20 bp, Artificial Sequence
  Primer
aggcttctgagcaggatgtc

SEQ ID NO: 72 - DNA, 19 bp, Artificial Sequence
  Primer
ggttggttatagcccaggt

SEQ ID NO: 73 - DNA, 24 bp, Artificial Sequence
  Primer
ggagaagattaacaggatgaagga

SEQ ID NO: 74 - DNA, 22 bp, Artificial Sequence
  Primer
gcttctgtgcttatgtctttgg

SEQ ID NO: 75 - DNA, 27 bp, Artificial Sequence
  Primer
ggaaaagtaagtatatggcatacagca

SEQ ID NO: 76 - DNA, 22 bp, Artificial Sequence
  Primer
agtcaatgcagttcttcaacac

SEQ ID NO: 77 - DNA, 23 bp, Artificial Sequence
  Primer
ggcaagagacaaaaagaaaactg

SEQ ID NO: 78 - DNA, 19 bp, Artificial Sequence
  Primer
gctcccggagtactcaacc

SEQ ID NO: 79 - DNA, 24 bp, Artificial Sequence
  Primer
cacagagtaggacataggtgacca

SEQ ID NO: 80 - DNA, 26 bp, Artificial Sequence
  Primer
gattaaatcaatagaggcaatcttcc

SEQ ID NO: 81 - DNA, 21 bp, Artificial Sequence
  Primer
caagcacacctctcaatctcc

SEQ ID NO: 82 - DNA, 24 bp, Artificial Sequence
  Primer
gttgcttagtatcagaatggtgga

SEQ ID NO: 83 - DNA, 26 bp, Artificial Sequence
  Primer
tcagtagtctgacatccaagaactgt

SEQ ID NO: 84 - DNA, 21 bp, Artificial Sequence
  Primer
ccttaatgtgggttgaagcac

SEQ ID NO: 85 - DNA, 24 bp, Artificial Sequence
  Primer
ggtactgacacaggataagagcag

SEQ ID NO: 86 - DNA, 20 bp, Artificial Sequence
  Primer
gagaagcggacaatcttgga

SEQ ID NO: 87 - DNA, 20 bp, Artificial Sequence
  Primer
gcctttttctgaacccatga

SEQ ID NO: 88 - DNA, 20 bp, Artificial Sequence
  Primer
agggccttaatatgggctgt

SEQ ID NO: 89 - DNA, 18 bp, Artificial Sequence
  Primer
tgtccggttcctcatcca

SEQ ID NO: 90 - DNA, 21 bp, Artificial Sequence
  Primer
tgaaggaaaccaacactgaca

(19) United States
(12) Patent Application Publication
(10) Pub. No.: US 2015/0087610 A1
(43) Pub. Date: Mar. 26, 2015

(54) Method of Modifying Fertility

(71) Applicant: Monell Chemical Senses Center, Philadelphia, PA (US)

(72) Inventors: Liquan Huang, Havertown, PA (US); Jiang Xu, Havertown, PA (US)

(73) Assignee: Monell Chemcial Senses Center, Philadelphia, PA (US)

(21) Appl. No.: 14/386,603

(22) PCT Filed: Mar. 18, 2013
(86) PCT No.: PCT/US2013/032767

§ 371 Date: Sep. 19, 2014

Related U.S. Application Data
(60) Provisional application No. 61/613,397, filed on Mar. 20, 2012.
(60) Provisional application No. 61/616,382, filed on Mar. 27, 2012.
(60) Provisional application No. 61/696,577, filed on Sep. 4, 2012.

(51) Int. Cl. A61K 31/522; A61K 31/17; A61K 31/167; A61K 31/365; A61K 31/7034; G01N 33/50; A61K 31/513

(52) U.S. Cl. CPC A61K 31/522; G01N 33/5044; G01N 33/5041; A61K 31/17; A61K 31/513; A61K 31/365; A61K 31/7034; A61K 31/167
USPC 514/35; 435/375; 435/7.21; 506/10; 514/263.34; 514/585; 514/274; 514/450; 514/620

(57) ABSTRACT

The present invention provides methods and compositions for modifying fertility in a male mammalian subject by contacting the subject's testis cells, germ cells or sperm with a sufficient amount of a composition comprising a ligand that binds, activates, or inhibits activation of, a TAS2R receptor expressed on the cells. Also described are methods for screening a test molecule for its effect on fertility by examining changes in male germ cells, testis cells or sperm resulting from contact with a molecule that binds, activates, or inhibits activation of, a TAS2R receptor expressed on the cells. Compositions for modifying fertility in a mammalian subject, e.g., contraceptive products, include a ligand that binds, inhibits or activates a TAS2R receptor in a pharmaceutically acceptable carrier.

### STATEMENT REGARDING FEDERALLY SPONSORED RESEARCH OR DEVELOPMENT

This invention was made with government support under Grant Nos. RO1 DC007487, P30 DC011735 and DBI-0216310 awarded by the National Institutes of Health and National Science Foundation. The government has certain rights in this invention.

### INCORPORATION BY REFERENCE OF MATERIAL SUBMITTED IN ELECTRONIC FORM

Applicant hereby incorporates by reference the Sequence Listing material filed in electronic form herewith. This file is labeled “MON6PCT_ST25.txt”, was created on 16 Mar. 2013, and is 16.0 KB.

### BACKGROUND OF THE INVENTION

Human fertility is a worldwide problem. On the one hand, infertility or subfertility, particularly male subfertility, is becoming increasingly common. Assisted conception including in vitro fertilization (IVF) and intra-cytoplasmic sperm injection is expensive, invasive and unreliable, and may cause epigenetic changes. On the other hand, the need to develop safe, simple, effective and reversible contraceptives is overwhelming. To address these two problems we face today, it is important to understanding the processes of spermatogenesis and sperm maturation, and their interactions with internal and external factors.

Mammalian spermatogenesis and sperm maturation include at least three phases: 1) mitotic: stem cells in the testis differentiate into spermatogonia, which undergo a limited number of mitoses; 2) meiotic: diploid spermatogonia enter meiosis, each of them giving rise to four haploid spermatocytes; and 3) postmeiotic: haploid spermatocytes undergo the most dramatic morphological changes and transform into immature spermatozoa, which are released into the lumen of seminiferous tubules of the testis. Sperm then further mature in the epididymis and fuse with prostasomes derived from the prostate glands before being ejaculated. In the female reproductive tract, the sperm are capacitated and hyperactivated, and finally one of them initiates the acrosome reaction and fuses with the egg.

Each phase of mammalian spermatogenesis consists of many critical cellular and molecular steps, many of which are susceptible to the influence of intrinsic and extrinsic factors. For example, intrinsically, the testis expresses tissue specific or cell differentiation stage-specific genes or splicing variants that are found only in spermatogonia and spermatocytes and stored in spermatids. Mutations in at least 200 genes can adversely affect mammalian sperm production and function. Extrinsically, environmental agents such as pesticides, phytoestrogen, heavy metals, and other toxic compounds can significantly contribute to the development of infertility and subfertility, and possibly also to epigenetic changes.

Bitter taste receptors (e.g., taste receptors type 2 or T2rs) were initially identified from taste bud cells in the oral cavity. Activation of these receptors triggers an innate aversion response. Since many bitter-tasting compounds are potentially toxic, these receptors seem to provide warning signals against ingestion of these poisons. However, some of these bitter compounds appear to have health benefits and possibly of hedonic valence as well. Bitter-tasting food and drinks such as certain vegetables, chocolate, coffee, beer, and tea are consumed and even liked by adult humans. Interestingly, T2r receptors are also expressed in the gastrointestinal tract, and thus the ingested bitter-tasting compounds are continuously monitored along the intestines, although the exact functions of these receptors in these tissues are yet unknown. T2r receptors are also found in the solitary chemosensory cells in the nasal cavity and lung, and possibly in other cells of the respiratory system, suggesting that these receptors may play a role in removing potentially harmful substances and initiating protective responses. Moreover, bitter taste receptors are reported to be present in the central nervous system, suggesting a possible role of these receptors in detecting internal toxic compounds.

Currently, the molecular mechanisms underlying spermatogenesis, sperm maturation and fertilization are not fully understood. Therefore, the treatment for infertility or subfertility is based on incomplete knowledge. There remains a need in the art for methods and compositions for modifying fertility.

### SUMMARY OF THE INVENTION

In one aspect, a method of modifying fertility in a male mammalian subject involves contacting the subject's testis cells, germ cells or sperm with a sufficient amount of a composition comprising a ligand that binds, activates, or inhibits activation of, a TAS2R receptor expressed on the cells. The contacting can involve in vivo administration of the ligand to a male subject. Alternatively, the contacting between the ligand and the receptor expressed in or on the cells or sperm occurs ex vivo and includes treating a sample of a mammalian subject's sperm with a sufficient amount of the ligand. The TAS2R receptors selected for use in this method are those of the mammal being treated or homologs or orthologs thereof. Similarly the ligands selected are those that bind the specific TAS2R receptor(s), homologs, and orthologs for the mammalian subject and either inhibit or activate the receptor. In one embodiment, the method results in an increase of the quality and/or quantity of sperm produced by the subject. In another embodiment, the method results in a decrease of the quality and/or quantity of sperm produced by said subject.

In another aspect, a method of modifying fertility in a female mammalian subject involves contacting sperm cells present in the reproductive system of a female prior to fertilization with a sufficient amount of a ligand that inhibits or activates, TAS2R receptors expressed on the sperm cells. The TAS2R receptors selected for use in this method are those of the mammal being treated or homologs or orthologs thereof. Similarly the ligands selected are those that bind the specific TAS2R receptor(s), homologs, and orthologs for the mammalian subject and either inhibit or activate the receptor. In one embodiment, the method results in an increase of the quality and/or quantity of sperm. In another embodiment, the method results in a decrease of the quality and/or quantity of sperm. In one embodiment, the ligand may be administered prior to or during insemination or IVF treatment to enhance the chances of conception. In another embodiment, the ligand is administered intravaginally prior to fertilization, such as by a douche, vaginal treatment, or contraceptive treatment, to reduce fertility.

In yet a further aspect, a method for screening a test molecule for its effect on fertility includes contacting a mammalian sperm cell, testis cell or cell line expressing a TAS2R receptor ex vivo with a test molecule; and assaying the contacted cells or cell lines for a change in a characteristic or function (e.g., activity) of the contacted receptor in comparison with a reference cell or cell line contacted with a control molecule. A change in a physical or functional characteristic of the test cells or cell lines vs. the reference indicates a modifying effect of the test molecule on the quality and/or quantity of sperm. In one embodiment, the assay detects a change in the cytosolic calcium concentration indicative of TAS2R receptor activation in comparison with a reference cell or cell line contacted with a control molecule. An increase or decrease in cytosolic calcium concentration of the test cells or cell lines vs. the reference indicates a modifying effect of the test molecule on the quality and/or quantity of sperm. In one embodiment, the method is a high-throughput method comprising multiple cells, cell lines, test molecules and references. In another embodiment, the assaying comprises an imaging assay or other known assay technique.

In another aspect, a composition for modifying fertility in a mammalian subject comprises a ligand that binds, inhibits or activates a TAS2R receptor in a pharmaceutically acceptable carrier. In one embodiment, the composition enhances fertility. In another embodiment, the composition is a contraceptive, such as a ligand-coated condom.

In yet another aspect, the use of comprises a ligand that binds, inhibits or activates a TAS2R receptor for the modification of mammalian fertility, either enhancement of fertility or contraception, is provided.

Other aspects and advantages of the invention will be readily apparent from the following detailed description of the invention.

### BRIEF DESCRIPTION OF THE DRAWINGS

FIG. 1A is an electrophoretic gel photograph showing Tas2r gene expression in testis. Reverse transcription-PCR covering nearly full coding sequences was performed for several Tas2r genes with cDNA template reverse transcribed from mouse testis poly (A)+RNA in the presence (+) or absence (−) of reverse transcriptases. The identities of the amplified products were confirmed by sequencing analysis.

FIG. 1B is a bar graph showing quantitative real time-PCR results from an assay conducted with cDNA prepared from mouse testis poly(A)+RNA. Transcripts for all 35 known Tas2rs were detected. An averaged transcript copy number per nanogram of poly (A)+RNA was obtained from three independent experiments with three mice.

FIG. 2 is a schematic showing the generation and breeding of Tas2r105-eGFPcre transgenic mice with two reporter strains. The transgene was generated using E. coli recombination system. A major portion of Tas2r105 gene in a bacterial artificial chromosome clone was replaced by IRES-eGFPcre-FRT-kan-FRT cassette. The kanamycin-resistant gene (kan) was flipped out using flp recombinases. After validation of the sequence, the transgene was microinjected into fertilized mouse eggs. The transgenic mice, which expressed eGFPcre under the Tas2r 105 promoter, were bred with the reporter strains ROSA26-lacZ and ROSA26-DT A.

FIG. 3A demonstrates that activation of spermatids by bitter tastants increases intracellular calcium concentrations. This figure is a graph showing a typical calcium response trace from the head area of a male germ cell. Spermatids were loaded with the calcium-sensitive dye Fura-2AM (inset: an image of a representative spermatid). A mixture of bitter taste (BTM) compounds of caffeine, N-phenylthiourea (PTC), 6-propyl-2 thiouracil (PROP), picrotin, salicin and denatonium (200 μM each) was first used to identify responsive spermatids, followed by a series of concentrations of the mixture.

FIG. 3B is a graph showing a dose response curve from 13 responsive cells described in FIG. 5A with an EC₅₀ was 13±11 μM.

FIG. 4A is a graph showing that the single bitter tasting compound picrotin activates spermatids. Traces from three representative cells are shown in response to a series of picrotin concentrations. No responses were detected to 0.01, 0.1 and 1 μM picrotin. Thus the shorter intervals were applied between these stimuli than those between higher concentrations.

FIG. 4B is a dose-response curve plotted with the data from 20 and 44 responsive cells to picrotin, with calculated at the concentration of half maximal effect (EC₅₀) values of 20±10 μM.

FIG. 4C is a graph showing that the single bitter tasting compound PROP activates spermatids. Traces from three representative cells are shown in response to a series of PROP concentrations. No responses were detected to 0.01, 0.1 and 1 μM PROP. Thus the shorter intervals were applied between these stimuli than those between higher concentrations.

FIG. 4D is a dose-response curve plotted with the data from 20 and 44 responsive cells to PROP, with calculated EC₅₀ values of 24±12 μM.

FIG. 5A is a graph showing trace of calcium responses from acrosome (A), midpiece (M) and principal piece (P) of a spermatid. Spatiotemporal characterization of bitter-tasting compounds evoked calcium signaling in mouse male germ cells. Inset is a Fura-2 am-loaded spermatid.

FIG. 5B is a graph showing trace of calcium responses from acrosome (A), midpiece (M) and principal piece (P) of a epididymal sperm. Spatiotemporal characterization of bitter-tasting compounds evoked calcium signaling in mouse male germ cells. Inset is a Fura-2 am-loaded epididymal sperm.

FIG. 6 is a graph showing traces of five epididymal sperm cells in response to denatonium, PROP, PTC, cycloheximide and salicin tastants. Individual male germ cell exhibited different ligand-activation profiles. Cell 1 responded to the first four compounds with similar intensity. Cell 2 responded only to cycloheximide. Cell 3 responded to cycloheximide and weakly to PROP. Cell 4 responded weakly to denatonium, PROP and cycloheximide. Cell 5 responded weakly to denatonium and cycloheximide. Cells responsive to salicin were rare and not shown.

FIG. 7A is a response trace from a representative sperm showing that calcium responses of mouse sperm were suppressed by the bitter blocker and abolished by the Gnat3 gene knockout. The increase in the intracellular calcium concentration was significant in response to 300 μM N-phenylthiourea 1 (PTC) 2 alone, nearly undetectable after the incubation with 1 mM probenecid, and partially recovered after the wash-off of probenecid (arrows).

FIG. 7B is a bar graph showing the quantification of the peak responses from 26 sperm at the three different time points described in FIG. 7A. Values are mean±SE.

FIG. 7C is a response trace showing that probenecid (1 mM) has no inhibitory effect on the response to cycloheximide.

FIG. 7D is a trace response showing that sperm isolated from the Gnat3−/−mutant mice did not respond to 2 mM salicin, PTC, denatonium (Dena), 6 propyl-2-thiouracil (PROP), cycloheximide (Cyclo), but 7 did respond to 2 mM ATP.

### DETAILED DESCRIPTION OF THE INVENTION

The methods and compositions described herein are based upon the inventors' discovery that many bitter taste (T2R or TAS2R) receptors are expressed in male germ cells. Male germ cells, like taste bud cells in the oral cavity and solitary chemosensory cells in the airway, are theorized to use TAS2R receptors to detect and respond to potentially poisonous compounds in the environment. Activation by strong agonist or inhibition by bitter blockers can manipulate the quality and quantity of sperm as well as affect the fertilization process. As supported by the data of the examples, the inventors have identified new infertility and subfertility treatments and contraceptive measures, and compositions.

As described in the examples herein, all 35 known bitter taste receptors were found to be expressed in mouse testis, particularly in the postmeiotic haploid germ cells in mammalian seminiferous tubules. Transgenic studies indicated that the majority of spermatogenic cells express these receptors. Individual male germ cells express postmeiotically most, but not all, TAS2R genes. Individual male germ cells, spermatids and spermatozoa expressed distinct subsets of TAS2R receptors and may display different bitter ligand-activation profiles.

Binding, inhibition or activation of these receptors by bitter-tasting compounds induces an increase in intracellular calcium concentration. Calcium signaling is an important cellular process in the spermatogensis and sperm maturation. Increase in cytosolic calcium concentrations induced by TAS2R receptor activation may alter other cellular functions such as morphological change, DNA or histone modification and chromatin packaging, leading to the reduced sperm function or epigenetic alterations. Such binding, inhibition or activation of these TAS2R receptors in male germ cells causes a decrease or an increase in sperm quantity and quality. Wild-type spermatids and spermatozoa responded to both naturally occurring and synthetic bitter tasting compounds by increasing intracellular free calcium concentrations. Overall, during the maturation from spermatids to epididymal sperm, the responsiveness of each cell to various bitter compounds increases, although individual cells may still have similar but distinct ligand-activation profiles

These data shed new light on the gene expression in male germ cells and suggested novel molecular mechanisms underlying the detection and response of spermatogenic cells and sperm to the environmental factors. These discoveries thereby provide novel methods and compositions for sub-fertility or infertility treatment and safe and reliable contraceptive methods and compositions in humans, as well as in animal breeding and pest animal control. These methods and compositions also provide assays for testing bitter taste sensitivity of animals or humans with polymorphisms alternative to expensive oral testing or cumbersome heterologous expression.

### I. DEFINITIONS AND COMPONENTS OF THE METHODS

All scientific and technical terms used herein have their known and normal meaning to a person of skill in the fields of biology, biotechnology and molecular biology and by reference to published texts, which provide one skilled in the art with a general guide to many of the terms used in the present application. However, for clarity, the following terms are defined as follows:

By “mammalian subject” is meant primarily a human, but also domestic animals, e.g., dogs, cats; livestock, such as cattle, pigs, etc.; common laboratory mammals, such as primates, rabbits, and rodents; and pest or wild animals, such as deer, rodents, rabbits, squirrels, etc.

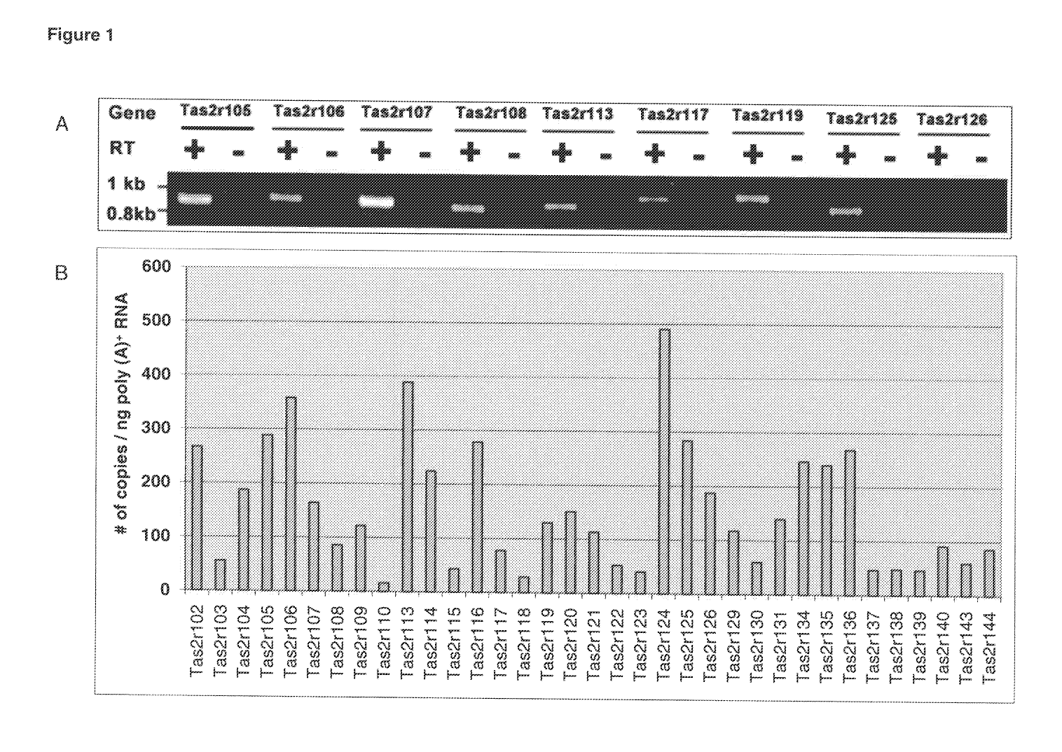

By “Tas2r” or “TAS2R” receptor is meant a bitter taste receptor located on mammalian male germ cells, and homologs and orthologs thereof. Humans have 25 TAS2R receptors; mice have 35 Tas2rs (see FIG. 1B); rats have 37 Tas2rs. Due to the different ecological niches that rodents and humans occupy, their bitter tastes have evolved differently. Thus, in one embodiment, a single mouseTas2r mouse has a single human counterpart. In another embodiment, a single mouse Tas2r corresponds to multiple human TAS2Rs and/or a single human TAS2R corresponds to multiple mouse Tas2rs. In still another embodiment, certain mouse Tas2rs have no human counterparts and is a species-specific receptor. As used herein, human receptors are capitalized, e.g., TAS2R1, while non-human receptors are indicated by lower case, e.g., mouse Tas2r102. When both human and non-human TAS2Rs are stated, the capitalized reference is used herein.

In one embodiment of the methods and compositions described herein, the receptor is TAS2R1. In another embodiment of the methods and compositions described herein, the receptor is TAS2R3. In another embodiment of the methods and compositions described herein, the receptor is TAS2R4. In another embodiment of the methods and compositions described herein, the receptor is TAS2R5. In another embodiment of the methods and compositions described herein, the receptor is TAS2R7. In another embodiment of the methods and compositions described herein, the receptor is TAS2R8. In another embodiment of the methods and compositions described herein, the receptor is TAS2R9. In another embodiment of the methods and compositions described herein, the receptor is TAS2R10. In another embodiment of the methods and compositions described herein, the receptor is TAS2R13. In another embodiment of the methods and compositions described herein, the receptor is TAS2R14. In another embodiment of the methods and compositions described herein, the receptor is TAS2R16. In another embodiment of the methods and compositions described herein, the receptor is TAS2R38. In another embodiment of the methods and compositions described herein, the receptor is TAS2R39. In another embodiment of the methods and compositions described herein, the receptor is TAS2R40. In another embodiment of the methods and compositions described herein, the receptor is, TAS2R41. In another embodiment of the methods and compositions described herein, the receptor is TAS2R42. In another embodiment of the methods and compositions described herein, the receptor is TAS2R43. In another embodiment of the methods and compositions described herein, the receptor is TAS2R44. In another embodiment of the methods and compositions described herein, the receptor is TAS2R45. In another embodiment of the methods and compositions described herein, the receptor is TAS2R46. In another embodiment of the methods and compositions described herein, the receptor is TAS2R47. In another embodiment of the methods and compositions described herein, the receptor is TAS2R48. In another embodiment of the methods and compositions described herein, the receptor is TAS2R49. In another embodiment of the methods and compositions described herein, the receptor is TAS2R50. In another embodiment of the methods and compositions described herein, the receptor is TAS2R60. These receptors are identified by structure in the Bitter DB website⁸² at the link http://bitterdb.agri.huji.ac.il/bitterdb/.

The term “homolog” or “homologous” as used herein with respect to any human TAS2R receptor means a nucleic acid sequence or amino acid sequence having at least 35% identity with the nucleotide or protein sequence, respectively, of a specific human TAS2R receptor used for comparison and encoding a gene or protein having substantially similar function to that of the reference sequence. Such homologs include mutants and variants of the T2r receptors 103-106 Such homologous sequences can be orthologs, e.g., genes in different species derived from a common ancestor. In other embodiments, the homolog can have at least 40, 50, 60%, 70%, 80%, 90% or at least 99% identity with the respective human sequence. In one embodiment, the homolog is that of a non-human mammalian species, e.g., such as the murine receptors identified in the examples below. Based on the known and publically available sequences of these receptors and the available computer programs readily available, such as the BLAST program, one of skill in the art can readily obtain full-length homologs, orthologs or suitable fragments of TAS2R genes or proteins thereof from a mammalian species.

By “ligand” as used herein is meant a compound that binds to a TAS2R receptor. In one embodiment, the ligand inhibits activation of a TAS2R receptor. In another embodiment, the ligand activates a TAS2R receptor. In certain embodiments, the same compound may activate one TAS2R receptor and modulate activation of another. Among such ligands are those identified in Meyerhof et al⁵³, in the Bitter database⁸², and other publications¹⁰⁷⁻¹⁰⁸ citedherein, including, without limitation, caffeine, N-phenylthiourea (PTC), 6-propyl-2 thiouracil (PROP), picrotin, salicin, quinine, denatonium benzoate, and absinthin.

Among such ligands are those that bind TAS2R1, including without limitation, lupulon, lupulone, humulon, humulone, arborescin, cascarillin, parthenolide diphenidol, diphenylthiourea, sulfocarbanilide, sym-diphenylthiourea, thiocarbanilide, thiamine, chloramphenicol, yohimbine, amarogentin, adhumulone, cohumulone, colupulone, isoxanthohumol, xanthohumol, dextromethorphan sodium, thiocyanate, trans-isohumulone, trans-isocohumulone, adlupulone, cis-isocohumulone, cis-isoloadhumulone, trans-isoadhumulone, sodium cyclamate, and picrotoxinin. In another embodiment, a ligand may be chloroquine that binds TAS2R 3. In another embodiment, a ligand for use in the methods and compositions is one or more of the ligands that binds TAS2R 4, including quinine, arborescin, artemorin, campher, parthenolide, quassin, azathioprine, chlorpheniramine, diphenidol, yohimbine, amarogentin, brucine, colchicine, dapsone, denatonium benzoate. In another embodiment, a ligand may be 1,10-phenanthroline that binds TAS2R 5.

In another embodiment, a ligand for use in the methods and compositions is one or more of the ligands that binds TAS2R 7, including quinine, caffeine, papaverine, chlorpheniramine, diphenidol, and cromolyn. In another embodiment, a ligand for use in the methods and compositions is one or more of the ligands that binds TAS2R 8, including: parthenolide, chloramphenicol, and denatonium benzoate. In another embodiment, a ligand for use in the methods and compositions is one or more of the ligands that binds TAS2R 9, including: ofloxacin, procainamide and pirenzapin. In another embodiment, a ligand for use in the methods and compositions is one or more of the ligands that binds TAS2R 10, including benzoin, quinine, absinthin, arborescin, arglabin, artemorin, caffeine, campher, cascarillin, coumarin, cucurbitacin B, papaverine, parthenolide, quassin, azathioprine, chlorpheniramine, diphenidol, haloperidol, chloramphenicol, yohimbine, dextromethorphan, chloroquine, picrotoxinin, thuj one, dapsone, denatonium benzoate, cucurbitacin E, cucurbitacins, cycloheximid, cycloheximide, erythromycin, strychnine, and famotidine.

In another embodiment, a ligand for use in the methods and compositions is one or more of the ligands that binds TAS2R 13, including diphenidol and denatonium benzoate. In another embodiment, a ligand for use in the methods and compositions is one or more of the ligands that binds TAS2R 14, including lupulon, lupulone, benzoin, quinine, absinthin, arborescin, arglabin, aristolochic acid, artemorin, caffeine, campher, cascarillin, coumarin, cucurbitacin B, falcarindiol, noscapine, papaverine, parthenolide, quassin, azathioprine, benzamide, carisoprodol, chlorhexidine, chlorpheniramine, diphenhydramine, diphenidol, diphenylthiourea, sulfocarbanilide, sym-Diphenylthiourea, thiocarbanilide, divinyl sulfoxide, flufenamic acid, haloperidol, sodium benzoate, isoxanthohumol, xanthohumol, trans-isohumulone, trans-isocohumulone, adlupulone, cis-isocohumulone, cis-isoloadhumulone, trans-isoadhumulone, chloroquine, 8-prenylnaringenin, picrotoxinin and thuj one.

In another embodiment, a ligand for use in the methods and compositions is one or more of the ligands that binds TAS2R 16, including diphenidol, sodium benzoate, amygdalin D, arbutin, helicon, D-salicin, sinigrin, salicin, phenyl beta-D-glucopyranoside. In another embodiment, a ligand for use in the methods and compositions is one or more of the ligands that binds TAS2R 38, including chlorpheniramine, diphenidol, diphenylthiourea, sulfocarbanilide, sym-diphenylthiourea, thiocarbanilide, yohimbine, sodium thiocyanate, sodium cyclamate, sinigrin, acetylthiourea, allyl isothiocyanate, caprolactam, dimethylthioformamide, ethylpyrazine, N-ethylthiourea, ethylene thiourea, N,N-ethylene thiourea, limonin, methimazole, 6-methyl-2-thiouracil, N-methylthiourea, phenethyl, isothiocyanate, phenylthiocarbamide (PTC), and propylthiouracil.

In another embodiment, a ligand for use in the methods and compositions is one or more of the ligands that binds TAS2R 39, including: quinine, chlorpheniramine, diphenidol, thiamine, chloramphenicol, amarogentin, chloroquine, colchicine, denatonium benzoate, acetaminophen. In another embodiment, a ligand for use in the methods and compositions is one or more of the ligands that binds TAS2R 40, including humulon, humulone, quinine, chlorpheniramine, diphenidol, adhumulone, cohumulone, colupulone, isoxanthohumol, xanthohumol and dapsone. In another embodiment, a ligand for use in the methods and compositions binds TAS2R 41 or TAS2R 42. In another embodiment, a ligand for use in the methods and compositions is one or more of the ligands that bind TAS2R 43, including quinine, arborescin, arglabin, aristolochic acid, caffeine, falcarindiol, diphenidol, chloramphenicol, amarogentin, denatonium benzoate, cromolyn, helicon, acesulfame K, aloin, grossheimin, saccharin. In another embodiment, a ligand for use in the methods and compositions is one or more of the ligands that bind TAS2R 44, including quinine, aristolochic acid, parthenolide, diphenidol, famotidine, acesulfame K, aloin, saccharin. In another embodiment, a ligand for use in the methods and compositions binds TAS2R 45.

In another embodiment, a ligand for use in the methods and compositions is one or more of the ligands that binds TAS2R 46, including quinine, absinthin, arborescin, arglabin, artemorin, caffeine, cascarillin, parthenolide, quassin, azathioprine, chlorpheniramine, diphenidol, chloramphenicol, yohimbine, amarogentin, picrotoxinin, brucine, colchicine, denatonium benzoate, strychnine, grossheimin, andrographolide, cnicin, crispolide, hydrocortisone, orphenadrine, tatridin B. In another embodiment, a ligand for use in the methods and compositions is one or more of the ligands that bind TAS2R 47, including absinthin, artemorin, campher, cascarillin, quassin, diphenidol, amarogentin, picrotoxinin, denatonium benzoate, andrographolide. In another embodiment, a ligand for use in the methods and compositions binds TAS2R 48. In another embodiment, a ligand for use in the methods and compositions is one or more of the ligands that binds TAS2R 49, including diphenidol or cromolyn. In another embodiment, a ligand for use in the methods and compositions binds TAS2R 50 such as amarogentin or andrographolide. In another embodiment, a ligand for use in the methods and compositions binds TAS2R 60.

The term “test molecule” as used herein can refer to any known or novel molecule for testing as a ligand that can bind, inhibit or activate a mammalian TAS2R receptor for safe use in humans and or other mammals. Such molecules may typically be found in known libraries of molecules, including those that have been pre-screened e.g., for safe use in animals. Suitable test molecules may be found, for example, in AMES library and may be readily obtained from vendors such as Otava, TimTec, Inc., Chem Bridge Corp., etc⁸³. The test molecules/compounds identified by the methods of this invention may be chemical compounds, small molecules, nucleic acid sequences, such as cDNAs, or peptides or polypeptides, which bind, inhibit or activate the TAS2R receptor in male germ cells, and affect the quantity and/or quality of the sperm produced. These test molecules may be used to modulate fertility as described herein. The compounds discussed herein also encompass “metabolites” which are unique products formed by processing the compounds of the invention by the cell or subject. Desirably, metabolites are formed in vivo.

“Male germ cell”, as used herein, refers to a sperm cell or one of its developmental precursors, including cells found in the testes of the male mammal, as well as cell lines created therefrom, spermatozoa and spermatids (collectively referred to as “sperm”).

By use of the term “native cell” is meant a mammalian cell or cell line that naturally or endogenously expresses the indicated Tas2r mRNA or protein receptor.

A “transformed cell or cell line” as used herein refers to a mammalian cell or cell line that is genetically engineered to express a desired TAS2R mRNA or protein that it does not naturally express or that it does not naturally express it in the known amounts. Particularly desirable cells or cell lines are selected from among any mammalian species, including, without limitation, cells such as A549, WEHI, 3T3, 10T1/2, HEK 293 cells, PERC6, Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention.

By “expression level” is meant the quantitative expression of the nucleotide sequence (e.g., mRNA) of a desired TAS2R or the quantitative expression of the desired expressed protein receptor itself.

The terms “control”, “reference”, “control subject” or “reference subject” are used interchangeably and refer to both an individual germ cell or germ cell line or the pooled data derived from multiple cells or cell lines or to numerical or graphical averages of the physical or functional changes in cells or cell lines. Such controls are the types that are commonly used in diagnostic assays for other receptors. Selection of the particular class of controls depends upon the use to which the diagnostic methods and compositions are to be put by the physician. As used herein, the term “predetermined control” refers to a numerical level, average, mean or average range of the characteristic, e.g., calcium signaling, of a TAS2R receptor in a defined male germ cell or male germ cell line population or a pattern of multiple changes for multiple physical or functional characteristics of the cell or cell line. The predetermined control level is preferably provided by using the same assay technique as is used for measurement of the effect of the test molecule on the cell or cell lines, to avoid any error in standardization. For example, the control may comprise a sample of sperm from one or more healthy male mammalian subjects contacted with a “control” compound, with which contact produces no physical or functional change. This reference or control can refer to a numerical average, mean or average range of the physical or functional characteristic being measured in the male germ cells or germ cell lines. It is also possible that the reference can be a sperm sample of the same subject, which is not contacted with the test molecule but with the inert control molecule.

The terms “a” or “an” refers to one or more, for example, “an assay” is understood to represent one or more assays. As such, the terms “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

### II. METHODS

In one aspect, therefore, a method of modifying fertility in a male mammalian subject comprises contacting the subject's testis cells, germ cells or sperm with a sufficient amount of a composition comprising a ligand that binds, activates, or inhibits activation of, a TAS2R receptor expressed on the cells. As discussed above, where the subject is a human, the mammalian TAS2R receptor is a human TAS2R receptor, including one or more of the receptors identified specifically above. Where the subject is a non-human animal, e.g., livestock for breeding, etc., the TAS2R receptor is an appropriate homolog or ortholog from a non-human mammal.

In one embodiment, contact between the ligand and the TAS2R receptor expressed in the germ cells of the subject inhibit the TAS2R receptor. In another embodiment, contact between the ligand and the TAS2R receptor expressed in the germ cells of the subject activates the TAS2R receptor. In one embodiment, such contact increases the quality and/or quantity of sperm produced by said subject and such method is desired wherein fertility in the subject is compromised or where an increase in fertility is desired, such as in animal breeding. In another embodiment, the contact of the ligand with the TAS2R receptor decreases the quality and/or quantity of male germ cell or sperm and is useful for contraception.

In circumstances in which the method involves a treatment for impaired fertility or infertility in the male subject, the contact occurs in vivo via administering the selected ligand for the selected receptor to a male subject. The selected ligand can include one or more of the ligands specifically identified above. Preferably these ligands are selected that have the characteristics of safe use in humans or animals. In certain embodiments, and depending upon the route of administration, a selected ligand is one that can pass the blood-testis barrier. Additionally, it may be desirable to administer more than one ligand to bind, inhibit or activate more than one receptor expressed in the male germ cells in vivo. The ligand selected increases the quality and/or quantity of sperm produced by said subject.

For male contraception, the ligand-containing composition may also be administered as a pharmaceutical composition. However, in this case the interaction between the selected TAS2R receptor and its selected ligand decreases the quality and/or quantity of sperm produced by said subject.

Thus, for administration in vivo, the ligand is desirably administered as a pharmaceutical composition as described below. The composition containing a ligand is desirably administered by any suitable route. In one embodiment, the ligand-containing composition is administered topically, such as by a cre\grave{m}e or ointment, or transdermal patch. In another embodiment, the ligand-containing composition is administered by intravenous injection at dosages sufficient to permit an effective amount to be delivered to the testis. In another embodiment, the ligand-containing composition is administered via an implant for timed release of suitable dosages. In this embodiment, the implant may be located close to the testis.

Depending upon the selection of the receptor, ligand, route of administration, the physical characteristics of the subject, and the purpose, e.g., enhancement of fertility or contraception, a suitable dosage of the ligand in the composition is between 1 nM to 100 mM ligand per kg body weight. In one embodiment, the dosage is at least 5 nM/kg. In another embodiment, the dosage is at least 10, 25, 50, 100, 200, 250, 300, 400, 500, 750, 800, or at least 900 nm/kg. In another embodiment, the dosage is at least 1 μM/kg. In one embodiment, the dosage is at least 5 μM/kg. In another embodiment, the dosage is at least 10, 25, 50, 100, 200, 250, 300, 400, 500, 750, 800, or at least 900 μM/kg. In another embodiment, the dosage is at least 1 mM/kg. In one embodiment, the dosage of ligand is at least 5 μM/kg. In another embodiment, the dosage is at least 10 mM/kg.

In yet another embodiment of the methods for modulating male fertility, the contacting occurs ex vivo and comprises treating or contacting a sample of a mammalian subject's sperm with a sufficient amount of the selected ligand. Such contact between the ligand and the TAS2R receptor can occur during collection, treatment, storage, transportation, or administration of sperm as part of an in vitro fertilization (IVF) procedure. For example, a suitable dosage of the ligand can be introduced into the medium in which the sperm is collected and used for insemination. Such ligands may protect the motility, survival, fertility capability and resistance to temperature and other changes to which sperm are subjected during fertility treatments.¹⁰⁹

In still another aspect, a method of modifying fertility in a female mammalian subject employs the selected TAS2R receptor ligands. In the case of the female subject, fertility can be modulated by administering to a female subject a sufficient amount of a ligand that inhibits or activates, TAS2R receptors expressed on sperm that are present in the reproductive system prior to fertilization. Thus to enhance fertility a suitable ligand is administered intervaginally prior to or during insemination or IVF treatment to enhance the chances of conception. Alternatively, the contact between the ligand and the sperm can occur any time before fertilization, and thus the ligand that can be introduced into the female reproductive system is present in a douche or vaginal treatment administered prior to or during insemination or IVF.

In another embodiment, the ligand can be present in a contraceptive composition introduced into the female reproductive system prior to fertilization, or prior to intercourse.

The embodiments for increasing fertility can be used for human IVF and also for animal breeding purposes. Particular TAS2Rs are selectively expressed in subpopulations of sperm, which provides a molecular basis for selectively breeding of animals with particular traits. Further, polymorphisms in TAS2R genes make it possible to personalize treatments.

The embodiments for decreasing fertility or contraception can be used for human contraception and for use in control animal populations, particularly pest populations. The differences in sensitivity to the selected TAS2R ligands among animal species enables one to design a ligand composition that can be used to differentially control one species (i.e., as a contraceptive) without affecting others. This is particularly advantageous in the wild.

### III. COMPOSITIONS

Still additional aspects of this invention are compositions for modifying fertility in a mammalian subject comprising a ligand that binds, inhibits or activates a TAS2R receptor in a pharmaceutically acceptable carrier. In one embodiment, the composition enhances fertility and increases the quality and/or quantity of sperm produced and is designed for in vivo use in a male subject. Such a composition may be a topical cream or ointment containing not only a suitable dosage of the ligand, but also conventional pharmaceutical excipients and other conventional ingredients. Another embodiment of a composition for administration to a male subject is a transdermal patch in which the ligand is embedded in a suitable dosage for topical delivery over a period of time. Still another embodiment is an implant that is designed to be located close to the testicles and which permits timed release in vivo of the ligand into the blood stream of the male subject.

Compositions for in vivo use in the female reproductive tract can be a rinse, douche or vaginal crème or ointment containing a ligand that increases the quality of sperm when used with IVF insemination or intercourse. Conventional pharmaceutical excipients and other conventional ingredients common to compositions for intravaginal use and not incompatible with the ligands are also provided by these compositions.

The compositions containing ligands described herein may be formulated neat or with one or more excipient for administration. One of skill in the art would readily be able to determine suitable excipients based on the selected TAS2R receptor, the mammalian subject, the selected ligand(s), the purpose for the composition, dosage needed and administration route, among others. Not only may the composition be solid or liquid, but excipient(s) may be solid and/or liquid carriers. The carriers may be in dry or liquid form and must be pharmaceutically acceptable. The compositions are typically sterile solutions or suspensions.

When the route of administration is intravenous injection or implant, the composition may be in the form of a liquid or suspension. When the route of administration is topical, such as transdermal patch, the composition may be additionally in the form of a crème. One of skill in the art would readily be able to formulate the compositions discussed herein in any one of these forms.

Suitable carriers and/or excipients include liquid carriers that may be utilized in preparing solutions, suspensions, and emulsions. In one embodiment, at least one TAS2R ligand is dissolved a liquid carrier. In another embodiment, at least one ligand is suspended in a liquid carrier. In one embodiment, the liquid carrier includes, without limitation, water, organic solvents, oils (such as fractionated coconut oil, arachis oil, corn oil, peanut oil, and sesame oil and oily esters such as ethyl oleate and isopropyl myristate), fats, cellulose derivatives such as sodium carboxymethyl cellulose.

Examples of excipients which may be combined with the ligands include, without limitation, antioxidants, binders, buffers, coatings, coloring agents, compression aids, diluents, disintegrants, emulsifiers, emollients, encapsulating materials, fillers, glidants, granulating agents, lubricants, metal chelators, osmo-regulators, pH adjustors, preservatives, solubilizers, sorbents, stabilizers, surfactants, suspending agents, thickening agents, or viscosity regulators. Other excipients may be used as listed in a variety of references^{94,95}. Specific examples of excipients for use in solid formulations include, without limitation, calcium phosphate, dicalcium phosphate, magnesium stearate, talc, starch, sugars (including, e.g., lactose and sucrose), cellulose (including, e.g., microcrystalline cellulose, methyl cellulose, sodium carboxymethyl cellulose), polyvinylpyrrolidine, low melting waxes, ion exchange resins, and kaolin. In one embodiment, the pharmaceutically acceptable excipient is a surfactant, binder, coating, disintegrant, filler, diluent, flavoring agent, coloring agent, lubricant, glidant, preservative, sorbent, sweetener, solubility increaser (such as cyclodextrans), analgesia enhancer (such as caffeine), or a combination thereof.

Finally, the ligands described herein can be added in suitable dosages to the culture media used to collect, store, transport or administer sperm for insemination or IVF procedures.

In yet other aspects, compositions containing one or more of the TAS2R ligands are designed for contraceptive use for humans or animals. In these contraceptive compositions the ligands used are those that decrease the quality and/or quantity of sperm produced by a male subject or present in a female reproductive tract prior to fertilization. One such embodiment is a contraceptive device which is impregnated with, or coated with, a suitable amount or dosage as described above, of one or more suitable ligands. For example, the ligand can be present in effective amounts and coated onto a condom designed for males or females. In another the ligand can be present in effective amounts in conventional contraceptive compositions, with or without additional contraceptives, such as spermicides. Similarly, the ligand can be coated onto a contraceptive implant or device, such as an IUD or contraceptive sponge.

### IV. SCREENING METHODS

Yet a further aspect provided by the inventors' discovery is a method for screening a test molecule for its effect on fertility. This method involves contacting ex vivo a mammalian germ cell(s) that expresses a TAS2R receptor from a single subject, particularly sperm, or another germ cell, or from a pool of multiple subjects, with a test molecule. One of skill in the art can also recombinantly generate a cell or cell line that expresses a selected TAS2R receptor(s) from a male germ cell or from a cell that normally does not express TAS2R by use of now-conventional techniques.⁸⁴ However, the ready availability of sperm samples would facilitate an assay for bitter taste receptor function in vitro in the absence of human subjects or animals, domestic or wild. The sperm cells can also be used to test the side effect of known medications used as test molecules in an assay on fertility, e.g., viability of sperm.

The test molecule is selected from among known or unknown ligands of TAS2R receptors, homologs or orthologs. Similarly the test molecule is selected from among libraries of molecules of unknown function or known medications to assess the effect of the test molecule on the physical or functional characteristics of normal sperm. The cells once contacted for a suitable time with a test molecule are then assayed for a change in a physical or functional characteristic of the cells in comparison with a reference cell or cell line contacted with a control molecule or contacted with nothing at all. A change in a physical or functional characteristic of the test cells or cell lines vs. the reference indicates a modifying effect of the test molecule on the quality or viability of the sperm.

For example, the contacted cell or cell line may be assayed for a change in the cytosolic calcium concentration indicative of TAS2R receptor activation in comparison with a reference cell or cell line contacted with a control molecule or with nothing. An increase or decrease in cytosolic calcium concentration of the test cell or cell line vs. the reference indicates a modifying effect of the test molecule on the quality and/or quantity of sperm. Other characteristics such as motility, viability, etc of sperm can also be evaluated in such an assay. In another embodiment, such assays can include electrophysiological responses or calcium imaging of isolated testes cells, cell lines or sperm cells.

These methods may also be high-throughput screening methods. In one embodiment such an assay involves contacting in each individual well of a multi-well plate a different selected test molecule (e.g., ligand, known or unknown molecules or drugs) with a mammalian germ cell or cell line (e.g., sperm) that expresses one or more TAS2R receptors. After the test molecule has been exposed to the sperm cell under appropriate culture conditions, a physical or functional characteristic of the sperm is conventionally measured. A change in a characteristic of the cell caused by any of the test molecules vs. the control permits the selection of the test molecule as one which enhances fertility or decreases fertility.

Such other assay formats may be used and the assay formats described herein are not a limitation. In one embodiment an acrosome reaction assay may be used. Whether a compound can increase or decrease acrosome reaction is important to the success of fertilization, particularly for IVF. Such assays are useful to identify useful TAS2R ligands, as well as to distinguish between those that enhance the quality, viability and/or quality of sperm from those that have the opposite effect. Other assays useful in these methods are immunocytochemistry assays, immunostaining assays, Western blots, TUNEL assays, cholesterol depletion assays, and cAMP concentration or activity assays, among others.

Another form of screening is to design a compound which has structural similarity to the test molecules identified herein by computational evaluation and a series of steps in which chemical entities or fragments are screened and selected for their ability to associate with or mimic known compounds or test molecules or ligands described herein. One skilled in the art may use one of several methods to screen chemical entities or fragments for their ability to mimic the structure of these peptides and more particularly to identify the structure that binds with the TAS2R receptor. This process may begin by visual inspection of, for example, a three dimensional structure on a computer screen. Selected fragments or chemical entities may then be positioned in a variety of orientations to determining structural similarities, or docked, within a putative binding site of the receptor.

Specialized computer programs that may also assist in the process of designing new test molecules based on those identified by the methods herein include the GRID program available from Oxford University, Oxford, UK.⁸⁵; the MCSS program available from Molecular Simulations, Burlington, Mass.⁸⁶; the AUTODOCK program available from Scripps Research Institute, La Jolla, Calif.⁸⁷; and the DOCK program available from University of California, San Francisco, Calif.⁸⁸, and software such as Quanta and Sybyl, followed by energy minimization and molecular dynamics with standard molecular mechanics force fields, such as CHARMM and AMBER. Additional commercially available computer databases for small molecular compounds include Cambridge Structural Database, Fine Chemical Database, and CONCORD database, among others⁸⁹.

Once suitable chemical entities or fragments have been selected, they can be assembled into a single compound. Assembly may proceed by visual inspection of the relationship of the fragments to each other on the three-dimensional image displayed on a computer screen in relation to the structure of the receptor. Useful programs to aid one of skill in the art in connecting the individual chemical entities or fragments include the CAVEAT program⁹⁰; 3D Database systems such as MACCS-3D database (MDL Information Systems, San Leandro, Calif.)⁹¹; and the HOOK program, available from Molecular Simulations, Burlington, Mass.

Compounds that mimic a test molecule described herein may be designed as a whole or “de novo” using methods such as the LUDI program⁹², available from Biosym Technologies, San Diego, Calif.; the LEGEND program⁹³ available from Molecular Simulations, Burlington, Mass.; and the LeapFrog program, available from Tripos Associates, St. Louis, Mo. Other molecular modeling techniques may also be employed^{96,97}. For example, where the structures of test compounds are known, a model of the test compound may be superimposed over the model of the target receptor. Numerous methods and techniques are known in the art for performing this step, any of which may be used⁹⁸⁻¹⁰². The model building techniques and computer evaluation systems described herein are not a limitation.

Thus, using these computer evaluation systems, a large number of compounds may be quickly and easily examined and expensive and lengthy biochemical testing avoided. Moreover, the need for actual synthesis of many compounds is effectively eliminated.

### V. EXAMPLES

Reverse transcription-PCR has isolated several Tas2r transcripts from mouse testis RNA, which were absent from the negative control sample prepared without reverse transcriptase. Quantitative real time-PCR has showed that the transcripts for all 35 known mouse Tas2rs were detected. In situ hybridization has localized the transcripts to haploid male germ cells. Transgenic studies using the promoter of mouse Tas2r105 gene has confirmed the receptor expression pattern, and furthermore, revealed that most but not all spermatogenic cells expressed Tas2r105.

Calcium imaging of mouse spermatids and epididymal sperm cells has demonstrated that the calcium responses to bitter tasting compounds are dose-dependent with EC₅₀ values close to those found with human T2R receptors. Individual cells may have different ligand-activation profiles; and overall responsiveness increases over the maturation process. Finally, immunostaining using an anti-human T2R antibody showed that human sperm cells also have bitter taste receptors.

### Example 1

### Methods and Materials

A. Reverse Transcription-PCR Analysis

To determine the expression of mouse Tas2R genes in the testis, total RNA was extracted from mouse testes using TRIZOL Reagent (Invitrogen). To enrich the target transcripts, poly (A)+RNA was isolated from the total RNA using Oligo(dT)25 Dynabeads (Invitrogen). One microgram of poly (A)+RNA was used as template to synthesize first strand cDNA with oligo (dT)15 primers and AMV DNA polymerase. A negative control was prepared with the omission of the DNA polymerase. The reaction mixtures of both the cDNA synthesis and negative control were diluted to 100 μL, of which 1 μL was used for each PCR reaction. PCR primers were designed to cover nearly entire Tas2r coding regions. PCR reactions were set up using FailSafe PCR System (Illumina), and PCR products were fractionated by agarose gel electrophoresis and confirmed by sequencing.

B. Quantitative Real Time-PCR Analysis

Real time PCR primers were designed and synthesized for all 35 predicted mouse Tas2Rs. PCR reaction was set up with FastStart TaqMan Probe Master (Roche Applied Science) and 1 μL of the diluted cDNA described above. The PCR reaction parameters were: 95° C. 10 min followed by 45 cycles of 95° C. 10 sec, 50° C. 15 sec and 72° C. 20 sec. PCR products were fractionated by agarose gel electrophoresis and confirmed by sequencing. The transcript copy number per nanogram of input poly(A)+RNA was calculated based on the cycle number at threshold (Ct) value against the standard curve plotted using a series of PCR reactions with the diluted mouse genomic DNA. The results from three independent experiments with three adult male mice were averaged (See FIGS. 1A and 1B).

C. In Situ Hybridization

Linearized plasmid DNAs containing 860 and 1013 bp of Tas2R105 and Tas2R108 receptor genes in pGEM-T Easy (Promega) and pCR4-TOPO (Invitrogen) vectors, respectively, were used to prepare ribonucleotide probes. Sense and antisense RNA probes were synthesized and digoxigenin-labeled from SP6, T3 and T7 promoters with DIG RNA labeling kit (Roche Applied Science). Tissue processing and probe hybridization were performed following the previously reported procedures with some modifications^{35,36}. Briefly, mouse testes were fresh frozen and sliced into 10 μm-thick sections, which were then fixed in 4% formaldehyde in 0.1 M PBS (pH 7.4) for 10 min. The sections were treated twice in 0.1% DEPC in PBS for 15 min, followed by washing 3 times in 5×SSC for 5 min. The sections were pre-hybridized in 5×SSC, 50% formamide, 50 μg/ml denatured sonicated salmon sperm DNA, 250 μg/ml yeast RNA, 1×Danhart's solution for 2 hours at 60° C. Adjacent sections were hybridized with 0.5-1 μg/ml DIG labeled antisense probes or antisense control probes. After overnight hybridization, the sections were washed at 68° C. in 0.1×SSC for 1 hour. Signals were detected using alkaline phosphatase-conjugated anti-digoxigenin antibodies and standard chromogenic substrates of 5-bromo-4-chloro-3-indolyl phosphate (BCIP) and nitroblue tetrazolium salt (NBT) (Roche Applied Science).

D. Generation of T2R105-GFPCre Transgenic Mice and Breeding with Rosa26-lacZ and DTA Mice

Bacterial artificial chromosome (BAC) clone RP23-342NS containing the entire mouse Tas2R105 gene was purchased from Children's Hospital Oakland-BACPAC Resources (http://bacpac.choriorg/). An IRES-eGFPcre-FRT-kan-FRT cassette was obtained from plasmid p1CGN21 and inserted into the mouse Tas2R105 gene in the BAC clone by replacing a DNA segment starting from 93 bp downstream of the start codon ATG to the last 60 bp upstream of the stop codon TGA of the mouse Tas2R105 through homologous recombination, followed by the flp recombinase-mediated removal of the kanamycin-resistant selectable marker gene using RED system. The correct sequence of the recombinant BAC clone was confirmed by DNA sequencing analysis. A large quantity of the BAC clone DNA was prepared, purified and microinjected into fertilized mouse eggs of C57BL/6J at the University of Pennsylvania Transgenic and Chimeric Mouse Facility.

The eggs were implanted into surrogate mothers and progeny were identified by PCR with the transgene-specific PCR primers (Sense primer: 5′-ACTTCAGATTCCCCCACAACA -3′ SEQ ID NO: 1, and antisense primer: 5′-TGCTTCCTTCACGACATTCAA-3′ SEQ ID NO: 2, with an expected PCR product of 384 bp). The transgenic mouse (mTas2R105-GFPcre) was bred with ROSA26-lacZ mouse (The Jackson Laboratory stock number 003474) to express the lacZ gene when its floxed upstream DNA stop sequence is excised by Cre recombinases. The transgenic mice were also bred to ROSA26-DTA mice to express diphtheria toxin fragment A in the cells where Cre recombinases are expressed.

E. LacZ Staining

Testes and tongues were collected from T2R5-eGFPcre transgenic mice, and fixed in 4% paraformaldehyde in phosphate-buffered saline (PBS) for 3 h on ice, and then cryoprotected in 20% sucrose in PBS overnight at 4° C. The tissues were embedded in Tissue-Tek® OCT compound (Sakura Finetek USA, Inc., Torrance, Calif.). Frozen sections (10 μm thickness) were obtained using a cryostat, and stored at −80° C. until use. The frozen sections were post-fixed with 4% paraformaldehyde for 30 minutes at room temperature, washed in PBS containing 0.02% NP40 (PBS-N) 3 times for 5 min each, and then rinsed with PBS. The sections were incubated with a pre-warmed PBS containing 0.1% 4-chloro-5-bromo-3-indolyl 3-D-galactopyranoside, 5 mM K₃Fe(CN)₆, 5 mM K₄Fe(CN)₆-3H₂0, and 2 mM MgCl₂ at 37° C. for 24 hours in the humidified chamber. After staining, the sections were stained with eosin for 4 min, and washed with running water.

F. Calcium Imaging with Testicular Cells and Epididvmial Sperm:

Eight adult male mice of each genotype: C57BL/6 and Gnat^{-/-} mice, were sacrificed, and the testis and caudal epididymis were immediately removed and transferred into HS solution containing (mM): 135 NaCl, 5 KCl, 2 CaCl₂, 1 MgCl₂, 30 Hepes, 10 glucose, 10 lactic acid, and 1 pyruvic acid (pH adjusted to 7.4 with NaOH) (Xia et al., 2007). For isolation of spermatids, seminiferous tubules from mouse testicle were dissected out and placed in a 15-mm dish containing 3 ml of HS medium. The tissue was finely minced and gently triturated with a fire-polished pipette, and the mixture was filtered with a 100 μm nylon cell strainer (BD Falcon, Bedford, Mass.). The dissociated cells were collected in a 1.5 ml plastic tube, and washed once in HS medium by centrifugation at 300 g for 4 min and resuspended in HS medium.

For isolation of epididymal spermatozoa, three incisions were made to the caudal epididymis, which was incubated in a 1.5-ml tube containing 1 ml of HS medium and 5 mg/ml bovine serum albumin (BSA) in a 5% CO₂ incubator at 37° C. for 20 minutes (Xia et al., 2007). Sperm cells released into the medium were collected from top 0.1 ml, which were adjusted to 1×10⁷ cells/ml HS medium.

Mouse spermatogenic cells were loaded with 5 μM acetoxymethylester of Fura-2 (Fora-2/AM) and 80 μg/ml pluronic F127 (Molecular Probes, Eugene, Oreg.) and transferred onto coverslips (22×60 mm, No. 0, Thomas Scientific Co.) for at least 1 hour at room temperature. The coverslip with spermatogenic cells was mounted in a recording chamber and superfused with HS or HS containing tasting compounds or the bitter blocker probenecid (Sigma-Aldrich, St. Louis, Mo.) via a valve controller (VC-8, Warner, USA).

Imaging of calcium responses was conducted as previously described (Gomez et al, 2005). Stimulation duration was 30 seconds and perfusion rate was 0.8 ml/min. Cells were excited at 340 nm and 380 nm and signals at 510 nm were captured by a cooled CCD camera. The change in fluorescence ratio (F=F₃₄₀/F₃₈₀) was recorded for regions-of-interest drawn on the cells.

G. Data Analysis

To analyze the dose-dependent calcium responses, the response intensity (F_{N}) was normalized and expressed as ΔF/F₀ value: ΔF/F₀=(F−F₀)/F₀. Here F₀ is the fluorescence at the beginning of stimulation, i.e., time 0, and F is the peak fluorescence ratio evoked by the stimulation. Dose-response curves and EC₅₀ values were generated based on the normalized responses using the software Origins by nonlinear regression.

H. Immunohistochemistry of Human Sperm

Human ejaculated sperm were purchased from California Cryobank and plated on a coverslip. Cells were fixed in 2% paraformaldehyde in PBS for 10 min, and washed with PBS three times, and blocked in 4% bovine serum albumin for 1 hour. A rabbit anti-human T2R receptor antibody was diluted in the blocking buffer and incubated with the coverslip overnight in the cold room. The binding of the primary antibody was visualized by an Alexa 488-conjugated donkey anti-rabbit secondary antibody. DAPi was used to stain nuclei acids.

### Example 2

### Mouse Testis Expresses the Bitter Taste Receptor Transcripts

To understand whether mammalian testis is able to detect and respond to potentially toxic, naturally occurring, or synthetic compounds, we set out to examine the expression of bitter taste receptors that are known to sense poisonous substances in the oral cavity and other organs. Reverse transcription-PCR with poly (A)+RNA prepared from mouse testis and Tas2r gene-specific primers (Table A) produced the expected PCR products that were absent in the control samples with the omission of the reverse transcriptases (FIG. 1A). Sequencing analysis of the amplified products confirmed their complete matches with mouse Tas2r105, 106, 107, 108, 113, 117, 119, 125 and 126 genes, respectively.

To determine the expression levels of all 35 identified mouse Tas2r genes, quantitative real-time PCR was carried out with cDNA templates prepared from poly(A)+RNA. Multiple pairs of PCR primers were designed and tested for each gene. Those that engendered specific and efficient amplification were used in the quantification experiments. (Table B). Since Tas2r genes are known to be intronless, a series of diluted mouse genomic DNA was used to obtain standard curves of Cₜ (threshold cycle number) value versus template copy number for all optimized pair of PCR primers, which were used to determine the transcript copy number of the target gene in the testis poly(A)+RNA samples. The averaged copy number of transcripts per nanogram of poly (A)+RNA from three mice varied from gene to gene (FIGS. 1A and 1B).

Based on these numbers, these genes can be largely classified into three categories:

1) abundant: consisting of 11 genes with their copy number of 200 or more, including Tas2r 102, 105, 106, 113, 114, 116, 124, 125, 134, 135 and 136;

2) medium abundant: consisting of 9 genes with transcript copy number ranging from 100 and 200, including Tas2r104, 107, 109, 119, 120, 121, 126, 129 and 131;

3) rare: consisting of the remaining 15 genes with transcript copy number below 100.

TABLE A
PCR PRIMERS FOR TAS2R CODING SEQUENCES

| | | FORWARD | REVERSE | |
|---|---|---|---|---|
| | | PRIMERS | PRIMER | |
| | | SEQ | SEQ | |
| | GENBANK | ID NOS: | ID NOS: | PRODUCT |
| TARGET | ACC. # | 3-11 | 12-20 | (BP) |
| Tas2r105 | NM_ | ATGCTGAGTG | CTAAAAGAACTTT | 903 |
| | 020501.1 | CGGCAGAAG | AATCCTTGCAGTA | |
| | | | CCTTTAC | |
| Tas2r106 | NM_ | ATGCTGACTG | CTACCATGTCACT | 927 |
| | 207016.1 | TAGCAGAAG | CTGACGTCCTT | |
| | | GAATC | | |
| Tas2r107 | NM_ | ATGCTGAATT | TTATTTGACTCTG | 927 |
| | 199154.1 | CAGCAGAAG | AAATTTCCTTTTG | |
| | | GCAT | TCTC | |
| Tas2r108 | NM_ | ATGCTCTGGG | CTACTTGTAGAAA | 894 |
| | 202050.1 | AACTGTATGT | CAGAAAATCTTCT | |
| | | ATTTGT | TTGCTTTAG | |
| Tas2r113 | NM_ | ATGGTGGCAG | CTATACTTCTATA | 930 |
| | 207018.1 | TTCTACAGAG | TTTTTAAACCTGT | |
| | | | ACCAGAG | |
| Tas2r117 | NM_ | ATGAAGCACT | CTAGAATATACA | 993 |
| | 207021.1 | TTTGGAAGA | ACATAATCTAAA | |
| | | | GTAC | |
| Tas2r119 | NM_ | ATGGAAGGTC | TTAAGATGGCATT | 1005 |
| | 020503.2 | ATATGCTCTT | ATACAGGCTTC | |
| | | CT | | |
| Tas2r125 | NM_ | ATGATGGGTA | TCAGGGAACCAA | 936 |
| | 207027.1 | TTGCCATAGA | CATCCGTA | |
| | | TATCTTA | | |
| Tas2r126 | NM_ | ATGCTACCAA | CTAGGCCACCCA | 927 |
| | 207028.1 | CATTATCAGT | GAATCC | |
| | | TTTCT | | |

TABLE B
REAL TIME-PCR PRIMERS FOR ALL 35 MOUSE TAS2R GENES

| | | FORWARD PRIMER | REVERSE PRIMER |
|---|---|---|---|
| | GENBANK | SEQ ID NOS: | SEQ ID NOS: |
| TARGET | ACC. # | 21-55 | 56-90 |
| Tas2r102 | NM_ | CTCCTGCTAATCTT | GGGTCTCTGTGTCTT |
| | 199153.2 | CTCTTTGTG | CTGG |
| Tas2r103 | NM_ | GGGTTCTTGGTATC | ACCATCCAGGAAAT |
| | 053211.1 | ATTATTGGAC | AGTAAGGAG |
| Tas2r104 | NM_ | GCAACACATCCTGG | CCCCATATTGGCAA |
| | 207011.1 | CTGAT | AAACAT |
| Tas2r105 | NM_ | AAGGCATCCTCCTT | GTGCAATAAATGTG |
| | 020501.1 | TCCATT | TTCCCTAAAA |
| Tas2r106 | NM_ | AGCCACATTCTTCT | AGCATGTAATGATA |
| | 207016.1 | CAACCT | GCCACCA |
| Tas2r107 | NM_ | GGCATCCTCCTTTG | TGCAATATATGTGT |
| | 199154.1 | TGTTGT | CCCCTAAAAC |
| Tas2r108 | NM_ | GTTTCTCCTGTTGA | GTGAGGGCTGAAAT |
| | 020502.1 | AACGGACT | CAGAAGA |
| Tas2r109 | NM_ | GTCAAATTCAGGTG | CACAGGGAGAAGAT |
| | 207017.1 | TTAGGAAGTC | GAGCAG |
| Tas2r110 | NM_ | CTTTCTCATGCTCA | GGCATCTCTAGGTG |
| | 199155.2 | TCTTCTCAC | GTTTGG |
| Tas2r113 | NM_ | CCACGGTAATGTTT | TGGTGCTGATGTCT |
| | 207018.1 | TCTTTGC | CTGCAT |
| Tas2r114 | NM_ | CGGCTGCCACTCAC | CAGCACTTTAATAG |
| | 207019.1 | TTATC | TTGCAGTATCATT |
| Tas2r115 | NM_ | CCTTTGGTGTATCC | CTGCATCTTCCTTAC |
| | 207020.1 | TTGATAGCTT | ATGTTTCA |
| Tas2r116 | NM_ | AAGGTTTGGAGTGC | AGCTGTTCTTGCAA |
| | 053212.1 | TCTGCT | CCTGTGT |
| Tas2r117 | NM_ | CCCTGTGGACACAT | TCACAGTTTGTAGG |
| | 207021.1 | CACAAG | GCTTTGAA |
| Tas2r118 | NM_ | CACTGGGTGCAGAT | CTTCAGAACAGTGA |
| | 207022.1 | GAAACA | ACTGAGCTTT |
| Tas2r119 | NM_ | AAGGAACCCAAGAC | AGGCTTCTGAGCAG |
| | 020503.2 | TCAGTGAC | GATGTC |
| Tas2r120 | NM_ | TGTTAACGAACTGG | GGTTGGTTATAGCC |
| | 207023.1 | CATTCAC | CAGGT |
| Tas2r121 | NM_ | CTGGTCTTATTGGA | GGAGAAGATTAACA |
| | 207024.1 | GATGATTGTG | GGATGAAGGA |
| Tas2r122 | NM_ | TCTTCTCTTTATGG | GCTTCTGTGCTTATG |
| | 001039128.1 | AGCCACCT | TCTTTGG |
| Tas2r123 | NM_ | CATTAAAGCCTTGC | GGAAAAGTAAGTAT |
| | 207025.1 | AAACTGTG | ATGGCATACAGCA |
| Tas2r124 | NM_ | CTCCACCATCATAC | AGTCAATGCAGTTC |
| | 207026.1 | TAATTGCAG | TTCAACAC |
| Tas2r125 | NM_ | AAGGCCTTGCACAT | GGCAAGAGACAAA |
| | 207027.1 | GGTAGT | AAGAAAACTG |
| Tas2r126 | NM_ | GTGTGTGGGATTGG | GCTCCCGGAGTACT |
| | 207028.1 | TCAACA | CAACC |
| Tas2r129 | NM_ | CAAAGATGCAGAGA | CACAGAGTAGGACA |
| | 207029.1 | TGTCCTTG | TAGGTGACCA |
| Tas2r130 | NM_ | TGCATTCATTGCAC | GATTAAATCAATAG |
| | 199156.1 | TGGTAAA | AGGCAATCTTCC |
| Tas2r131 | NM_ | TAGCCCACATTTCC | CAAGCACACCTCTC |
| | 207030.1 | CATCC | AATCTCC |
| Tas2r134 | NM_ | GCCTGGGAAGTGGT | GTTGCTTAGTATCA |
| | 199158.1 | AACCTA | GAATGGTGGA |
| Tas2r135 | NM_ | CCATCATGTCCACA | TCAGTAGTCTGACA |
| | 199159.1 | GGAGAA | TCCAAGAACTGT |
| Tas2r136 | NM_ | GGACAATGAGGCTT | CCTTAATGTGGGTT |
| | 181276.1 | TATGGAA | GAAGCAC |
| Tas2r137 | NM_ | CTGGCTCAAATGGA | GGTACTGACACAGG |
| | 001025385.1 | GAGCTT | ATAAGAGCAG |
| Tas2r138 | NM_ | CAAACCAAGTGAGC | GAGAAGCGGACAAT |
| | 001001451.1 | CTCTGG | CTTGGA |
| Tas2r139 | NM_ | AGTGCACCATAGGT | GCCTTTTTCTGAACC |
| | 181275.1 | ATCATTGC | CATGA |
| Tas2r140 | NM_ | GAAGAACATGCAAC | AGGGCCTTAATATG |
| | 021562.1 | ACAATGC | GGCTGT |
| Tas2r143 | NM_ | CATTGGCCTCTATG | TGTCCGGTTCCTCAT |
| | 001001452.1 | TTGCAG | CCA |
| Tas2r144 | NM_ | AAGCAGAAAATCAT | TGAAGGAAACCAAC |
| | 001001453.1 | AGGGCTGA | ACTGACA |

### Example 3

### Tas2R Transcripts are Localized to Postmeiotic Spermatids

To localize Tas2r transcripts to the expressing cells in the testis, in situ hybridization was perform on mouse testicular tubule sections with sense and antisense riboprobes for two representative Tas2r genes: one abundantly-expressed Tas2r105 and one rarely expressed Tas2r108. Antisense probes of Tas2r105 and Tas2r108 hybridized to subsets of cells in some seminiferous sections (data not shown), i.e., some cells of adjacent tubule sections whereas cells in other sections were not hybridized. High magnification images show that the stained cells were postmeiotic cells. No signals with the sense probes were detected.

Further examination of these seminiferous sections indicated that cells expressing Tas2Rs appeared to be the spermatocytes undergoing meiosis as well as those in the later stages of spermatogenesis. The negative controls of sense probes did not produce any specific or non-specific hybridization on the sections.

### Example 4

### Transgenic Studies Validate Tas2R Expression in the Testicular Cells

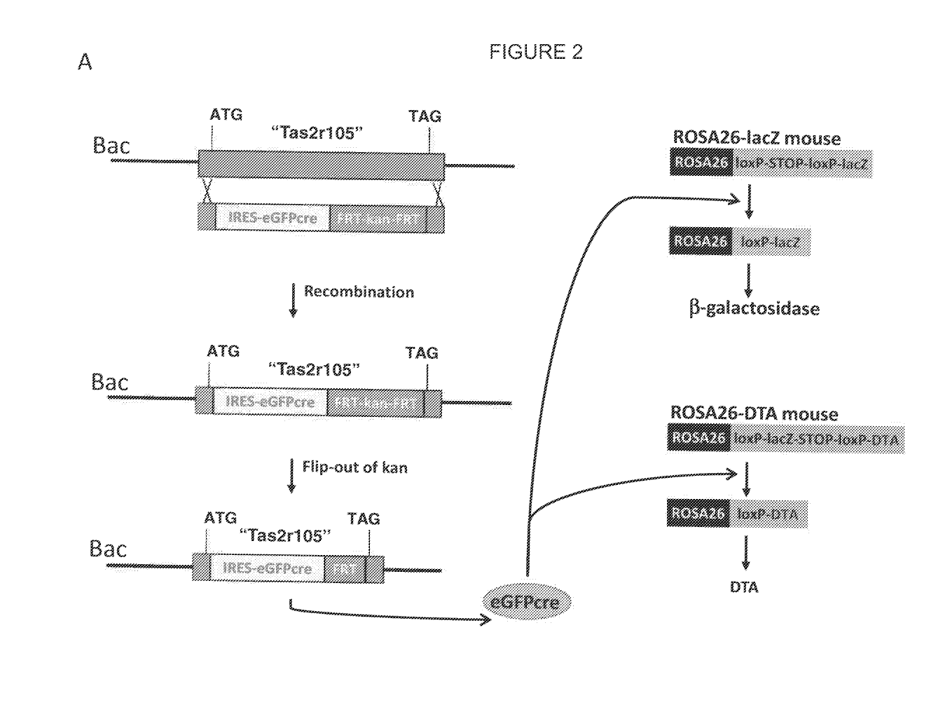

To confirm the expression pattern of mouse Tas2rs in the testis, we set out to generate transgenic animals. Bacterial artificial chromosome clone RP23-342N5, which contains 183 kilobase pairs of the mouse genomic DNA including the entire Tas2r105 gene, was used to construct the transgene (FIG. 2). An IRES-eGFPcre-FRT-kanFRT cassette was inserted into the Tas2r105 gene by replacing a segment of 750 bp of this gene's 903-bp coding sequence using a homologous recombination system in E. coli. After the kanamycin-resistant gene was flipped out with flp recombinase, the construct was injected into fertilized eggs of C57BL/6. The transgenic mice were identified by PCR with the genomic DNA isolated from tails and transgene-specific primers.

The green fluorescence in the testis of the transgenic mice, Tas2r105-eGFPcre, was too low to be detectable. To test the transgenic cre recombinase activity, the transgenic mice were bred with two reporter strains: 1) ROSA26-lacZ mice, which carry a flox-flanked stop sequence to prevent the expression of the downstream lacZ gene driven by the ROSA26 promoter³⁸; and 2) ROSA26-DTA, which carries the coding sequence for diphtheria toxin fragment A downstream of a floxed stop sequence directed by the ROSA26 promoter.

Examination of the adult male bi-transgenic Tas2r105-eGFPcre:ROSA26-lacZ mice indicated that while the size and weight of testis were comparable with those of the wild-type control, the β-galactosidase activity was detected in the bi-transgenic testis but not in those of the wild-type control mice (data not shown). Expression of lacZ gene in the testis of the bitransgenic Tas2r105-eGFPcre:ROSA26-lacZ mice was observed (data not shown). Postmeiotic cells located close to the lumen in nearly all seminiferous sections displayed the enzymatic activity of β-galactosidase. Ablation of spermatogenic cells in the testis of the bitransgenic Tas2r105-eGFPcre:ROSA26-DTA mouse were detected in photomicrographs of a wild-type testis of an adult C57BU6 mouse and a testis from an adult bitransgenic mouse. Hematoxylin and eosin (H&E) staining of the testicular sections of the control and bitransgenic mice at low and high magnifications were conducted (data not shown).

In contrast, the size and weight of the adult male bi-transgenic Tas2r105-eGFPcre: ROSA26-DTA mice were significantly smaller than those of the age-matched wild-type controls. The average length and weight of the bi-transgenic testis at age of 5 months were 6 mm and 0.07 g respectively, while those of the control were 8 mm and 0.2 g, respectively. Postmeiotic spermatogenic cells were depleted in most of tubules. Haematoxylin and eosin staining of the bitransgenic testicular tissue showed that haploid germ cells were eliminated in all but few seminiferous sections.

### Example 5

### Bitter Taste Compounds Elicit Calcium Responses from Spermatids and Spermatozoa in a Dose-Dependent Manner

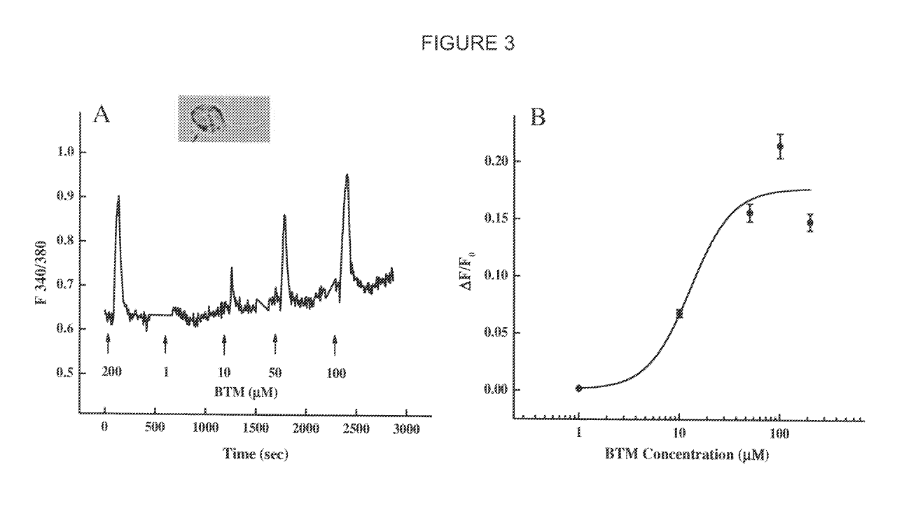

To examine whether mouse T2rs expressed in the testis are functional, we used a mixture of bitter tastants: caffeine, N-phenylthiourea (PTC), 6-propyl-2 thiouracil (PROP), picrotin, salicin and denatonium (200 μM each). Calcium responses were elicited from a number of freshly dissociated spermatids that were identified by their emerging tail and immature head structure (see inset of FIG. 3A). The responses were concentration-dependent within a range of 0.1 to 200 μM and a calculated EC₅₀ of 13±11 μM (see graphs of FIGS. 3A and 3B).

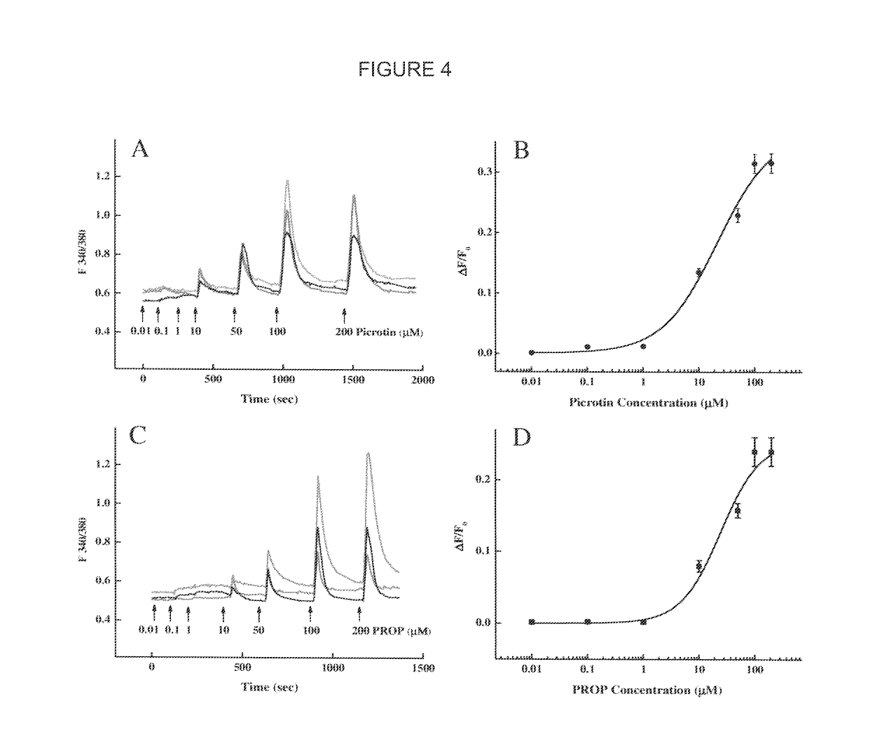

To test whether mouse spermatogenic cells can be activated by single bitter compounds, picrotin and PROP were used in the calcium imaging assays. The results showed that many mouse spermatids responded to picrotin or PROP in a dose dependent fashion with calculated EC₅₀ of 20±10 μM and 24±12 μM, respectively (see FIGS. 4A-4D).

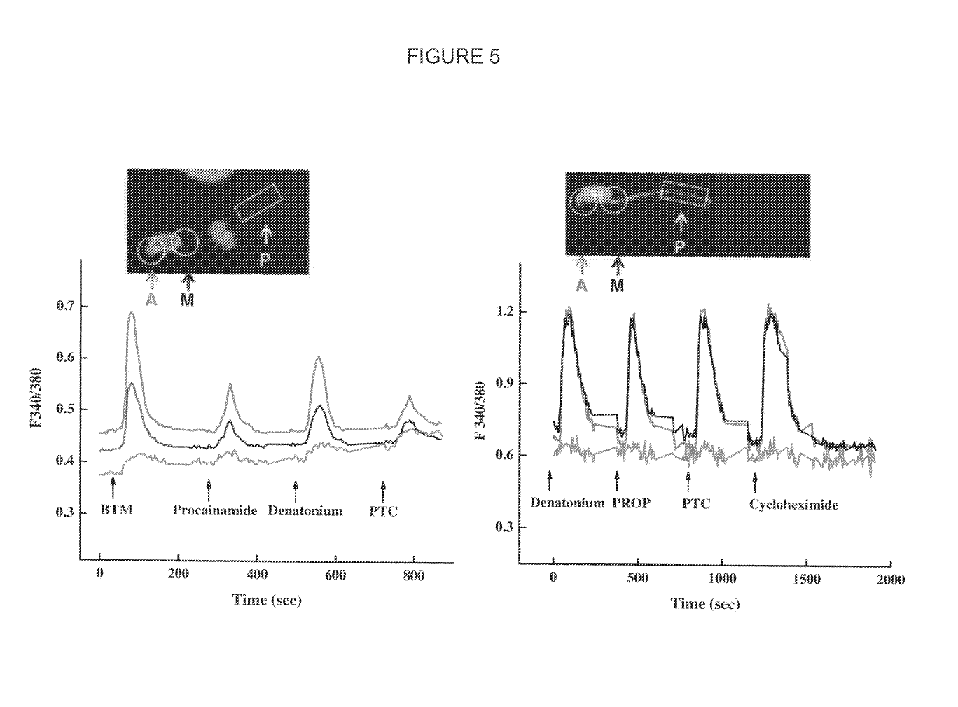

To determine whether there are subcellular differences in the calcium responses, areas of acrosome, midpiece and principal piece were monitored separately (FIGS. 5A and 5B). The increases in intracellular calcium concentrations in responses to a mixture of bitter compounds or individual compounds, procainamide, denatonium and PTC, were much larger in the acrosome than in the midpiece of mouse spermatids whereas the responses in the principal piece were detectable but much smaller than those in the other two areas (FIG. 5A). The initiation of the calcium response in these three areas, however, seemed to be simultaneous.

To reveal whether the subcellular response pattern is altered over the spermatozoon maturation, calcium imaging was performed with more mature sperm cells isolated from mouse cauda epididymis (FIG. 5B). The results showed that the amplitudes of the responses to denatonium, PROP, PTC and cycloheximide from the acrosome and midpiece were equally strong whereas the responses from the principal pieces were mostly undetectable, indicating that there was a shift in the distribution of bitter taste receptors and signaling components during this maturation period.

### Example 6

### Individual Germ Cells Exhibit Different Ligand-Activation Profiles

In taste bud cells, one receptor cell can be activated by multiple bitter compounds and the activation profiles are different from cell to cell. To characterize the response profiles of male germ cells, we selected five bitter compounds: denatonium, cycloheximide, PROP, PTC and salicin to stimulate mouse spermatids and epididymal sperm cells.

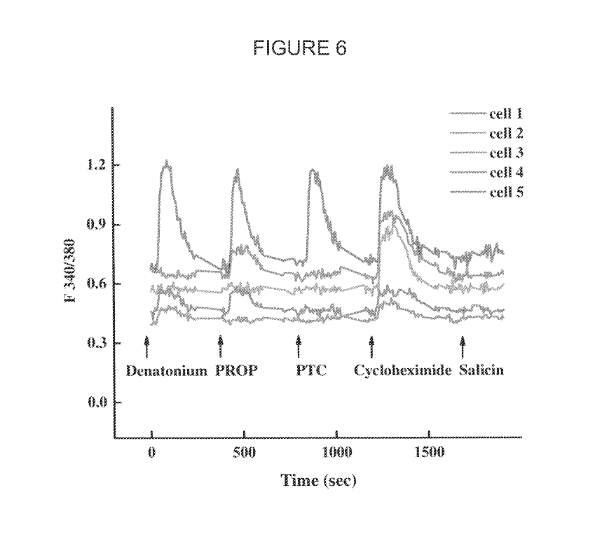

The results showed that individual cells displayed different response profiles (FIG. 6, Tables C and D). At least 10 and 13 different response profiles were found among testicular spermatids and epididymal sperm cells, respectively (Tables C and D). Some cells responded to all of the five compounds (Table D: profile 1), to 4 of these compounds (Table C, profile 1; and Table D, profiles 2, 3 and 4), to 3 of them (Table C, profiles 2 and 3; and Table D, profiles 5, 6, 7 and 8), to 2 of them (Table C, profiles 4,5 and 6; and Table D, profiles 9, 10 and 11). Other profiles responded to only one of these compounds (Table C, profiles 7, 8, 9 and 10; Table D, profiles 12 and 13). Some cells responded to different compounds with similar intensity whereas others exhibited different response intensity to the same batch of the bitter compounds (FIG. 6).

TABLE C
RESPONSE PROFILES OF INDIVIDUAL
SPERMATIDS TO BITTER COMPOUNDS

| | COMPOUNDS TO WHICH | | | | # CELLS |
|---|---|---|---|---|---|
| PROFILE | CELLS RESPONDED | | | | RESPONDED |
| 1 | Cyclo- | Denatonium | PROP | PTC | 3 |
| | heximide | | | | |
| 2 | Cyclo- | Denatonium | | PTC | 1 |
| | heximide | | | | |
| 3 | Cyclo- | | PROP | PTC | 1 |
| | heximide | | | | |
| 4 | Cyclo- | Denatonium | | | 7 |
| | heximide | | | | |
| 5 | Cyclo- | | PROP | | 1 |
| | heximide | | | | |
| 6 | | Denatonium | | PTC | 1 |
| 7 | Cyclo- | | | | 5 |
| | heximide | | | | |
| 8 | | Denatonium | | | 4 |
| 9 | | | PROP | | 3 |
| 10 | | | | PTC | 3 |

| | |
|---|---|
| TOTAL CELLS | 29 |

TABLE D
RESPONSE PROFILES OF INDIVIDUAL EPIDIDYMAL SPERM
CELLS TO BITTER COMPOUNDS

| PROFILE | COMPOUNDS TO WHICH CELLS RESPONDED | | | | | # CELLS RESPONDED |
|---|---|---|---|---|---|---|
| 1 | Cycloheximide | Denatonium | PROP | PTC | Salicin | 4 |
| 2 | Cycloheximide | Denatonium | PROP | PTC | | 13 |
| 3 | Cycloheximide | Denatonium | PROP | | Salicin | 1 |
| 4 | Cycloheximide | | PROP | PTC | Salicin | 1 |
| 5 | Cycloheximide | Denatonium | PROP | | | 4 |
| 6 | Cycloheximide | Denatonium | | PTC | | 1 |
| 7 | Cycloheximide | | PROP | PTC | | 1 |
| 8 | | Denatonium | PROP | PTC | | 1 |
| 9 | Cycloheximide | Denatonium | | | | 1 |
| 10 | Cycloheximide | | PROP | | | 5 |
| 11 | | Denatonium | | PTC | | 1 |
| 12 | Cycloheximide | | | | | 15 |
| 13 | | | | | Salicin | 1 |

| | |
|---|---|
| TOTAL CELLS | 49 |

Ligand-based analysis indicated that cycloheximide was the most effective of the five tested bitter compounds on the spermatids, inducing 18 out of 29 or 62.1% cells to increase their intracellular calcium concentrations. The second and third most effective compounds were denatonium and PROP, stimulating 55.2% and 27.6% cells to respond (Table C). Some spermatids responded to both of the N—C═S moiety-containing compounds PROP and PTC. Other spermatids responded to either or neither of the PROP and PTC. About half (51.7%) of the cells responded to a single compound only. No spermatids were found to respond to salicin (Table C).

Among the epididymal sperm cells, cycloheximide was the most effective compound as well and evoked the calcium responses from 46 out of 49 or 93.9% of the cells, whereas about 61.2% and 53.1% cells responded to the second and third most effective compounds PROP and denatonium, respectively. As with the spermatids, some epididymal sperm cells responded to both PROP and PTC, whereas others did to either or neither of the two compounds. In comparison with spermatids, fewer epididymal sperm cells (34.7%) responded to a single compound only. Further, about 14.3% of these cells were responsive to salicin.

### Example 7

### Human Sperm Express T2R Receptors

Immunostaining of human sperm with an anti-human T2R antibody (results not shown) showed that about 60% of sperm cells expressed this receptor, which is largely consistent with the expression pattern found in mouse sperm cells. Most receptors were shown to be concentrated in the midpiece with some residual proteins remaining in the head. Further examination of the stained sperm found that in the ejaculated sperm, most receptors are concentrated in the midpiece with some residual proteins remaining in the head. Since the ejaculated sperm contain fluid from prostate gland, these cells may have merged with such components as prostasomes from the gland and undergone further remodeling. Thus these cells are more mature than the ones we isolated from mouse tissues. It is likely that in earlier developmental stages, T2R receptors are more dispersed as found in the mouse cells. The finding of human T2R receptors in sperm cells indicates that these receptors may also be involved in human spermatogenesis, sperm maturation and fertilization.

### Example 8

### Germ Cells' Responses to Bitter Tastants can be Suppressed by a Bitter Blocker and Abolished by the α-Gustducin Gene Knockout

To confirm whether the germ cells' responses to bitter tastants were mediated by the bitter taste receptors, we examined the effect of the bitter taste receptor blocker probenecid (Greene et al, 2001) and nullification of the α-gustducin gene in Gnat3 α-gustducin knockout mice described in Wong et al, 1996. Probenecid is known to inhibit the responses of human T2R16, 38 and 43 to salicin, PTC and PROP, and aloin, but not that of T2R31 to saccharin (Greene et al, 2011).

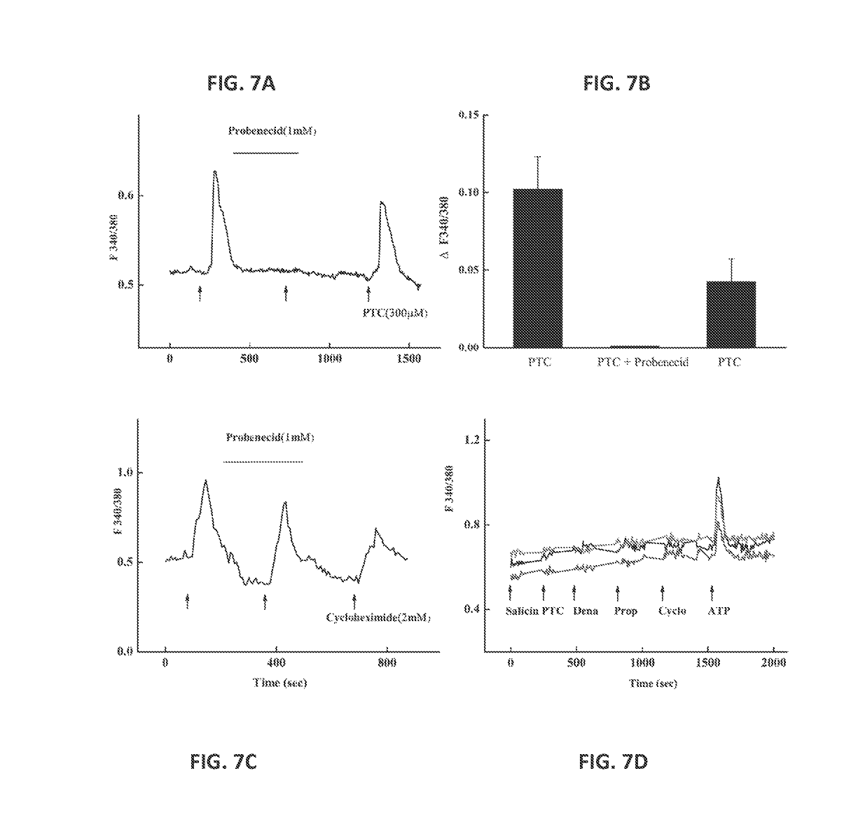

The results showed that preincubation of the C57B/6L wild-type sperm with 1 mM prebenecid for 5 min nearly completely suppressed the responses to 0.3 mM PTC (see FIGS. 7A and 7B). The suppression was reversible; and after the wash-off of the blocker, the cells' responses to PTC were restored although the amplitude was somewhat smaller than the pre-blocking ones. In contrast, probenecid did not block the responses to another bitter tastant, cycloheximide (FIG. 7C). Probenecid can also inhibit the mouse sperm's response to PTC, but not to cycloheximide (FIGS. 7A, 7B and 7C), suggesting that the mouse sperm's responses to bitter tastants were mediated byT2rs.

Calcium imaging of sperm isolated from the Gnat3−/−mutant mice which express no α-gustducin showed that these cells were unresponsive to the bitter tastants tested: salicin, PTC, denatonium, PROP and cyclohexmide at 2 mM, while the responses to 2 mM ATP seemed to be normal (Rodriguez-Miranda et al., 2008) (FIG. 7D). Thus, knock-out of the α-gustducin gene abolishes the sperm's calcium responses to several bitter tastants, indicating that spermatogenesis is affected by aTas2r-mediated signal transduction pathway.

The data in Examples 1-8 above provide evidence that Tas2r expression in haploid male germ cells provides a novel mechanism underlying the interaction of mammalian male germ cells with environmental toxicants. Bioinformatic analysis of mouse genome sequences has predicted about 35 genes encoding bitter taste receptors^{49,50}. As demonstrated in the examples above, the reverse transcription PCR results indicate that several mouse Tas2r genes were expressed in the testis. A full-scale interrogation using quantitative real-time PCR with primers for all 35 Tas2rs revealed that all these receptor genes were expressed in the tissue. Based on their expression levels, they can be roughly classified as abundant, medium abundant, and rare (see, e.g., FIGS. 1A and 1B).

In situ hybridization results localized the transcripts of two representative genes, Tas2r105 and 108, in postmeiotic cells (data not shown). Bitransgenic studies of Tas2r105-eGFPcre:ROSA26-lacZ demonstrated β-galactosidase activity in haploid germ cells, further supporting the notion of the Tas2r receptor expression in these spermatogenic cells (see, e.g., FIG. 2). These results, together with the Tas2r expression patterns in taste buds and solitary chemosensory cells, allow us to predict that the other 33 Tas2rs are likely expressed approximately at the same stage (i.e., the meiotic phase), although some Tas2rs may be expressed in the spermatogonia. However, these Tas2r genes are likely not transcribed in spermatozoa, since RNA synthesis ends before spermatids are released into the lumen⁵¹.

When Tas2r105-eGFPcre mice bred with ROSA26-DTA, the testes of their male offspring were smaller in size and weight (data not shown). H&E staining of the testis sections indicated that a vast majority of tubular sections lacked any haploid cells, and only a few sections seemed to have spermatocytes and spermatids as in the control sections. This strongly suggests that most mouse male germ cells express Tas2r105 at the meiotic phase; the reason for the absence of Tas2r105 in some cells is currently unknown. One possible explanation is that gene expression in meiotic cells may be subject to the microenvironment, which is not uniform throughout the organ.

To functionally characterize Tas2r receptors expressed in the male germ cells, we performed calcium imaging of the responses of acutely dissociated mouse testicular cells to bitter-tasting compounds. To help identify responsive cells from a heterogeneous pool of dissociated testicular cells, we initially applied a mixture of six bitter compounds (BTM): caffeine, PTC, PROP, picrotin, salicin, and denatonium. The response amplitude from the spermatids was concentration dependent, with an EC₅₀ of 13±11 μM, a large variation (FIGS. 3A and 3B) that may be at least partly attributable to the heterogeneity in the developmental stages of these germ cells dissociated from whole testis. Spermatids isolated from the same seminiferous location, with germ cells more likely at the same maturation stage and possibly expressing the same subset of Tas2rs, may exhibit a more homogeneous response.

The responsive cells identified with bitter compounds were tested with individual bitter-tasting compounds, picrotin and PROP. Some of these cells displayed a concentration-dependent response, with calculated EC₅₀ values of 20±10 μM to picrotin and 24±12 μM to PROP (see, e.g., FIGS. 6A-6D). Although it is unknown which mouse T2r receptors can be activated by picrotin, this compound can stimulate five heterologously expressed human T2rs, the most sensitive of which is T2r14, with an EC₅₀ value of 18 μM^{52,53}. This similarity in EC₅₀ values suggests that a mouse receptor orthologous to human Tas2R14 may exist in addition to other possible non-cognate receptors. Unlike picrotin, however, PROP can activate only one human bitter receptor, i.e., Tas2r38. Six variants of this receptor have been found: two unresponsive to PROP and four responding with EC₅₀ values of 2-4 μM^{54,55}.

Our data suggest that one or more less-sensitive receptors with EC₅₀ values of 24 μM may occur in rodent testicular spermatids. Calcium imaging results of multiple subcellular areas of the spermatids indicated that the increase in intracellular calcium concentrations seemed to be simultaneous, although the amplitudes of the calcium responses differed remarkably, with the acrosome, midpiece, and principal piece having highest, medium, and least response, respectively (see, e.g., FIGS. 5A and 5B). These results suggest that Tas2rs and their signaling components are distributed across the spermatid, with apparent concentrations in the head region. The response pattern in epididymal sperm differed: the responses from the acrosome and midpiece appeared equally large, while that from the principal piece became barely detectable (see, e.g., FIGS. 5A and 5B). This change may have resulted from the redistribution of the proteins within the sperm cell during maturation: the receptor protein and their downstream signal transduction components are more concentrated in the cytosol-rich midpiece. Restricted subcellular localization for some functionally specialized proteins has been reported. For example, a sperm-specific, calcium ion-permeable channel, CatSper, is located exclusively in the sperm tail⁵⁶.

Comparison of bitter tastant response profiles indicated that individual male germ cells may have different response profiles: some cells responded to more tastants than did others, and cells varied in response intensity (FIG. 6). Based on the limited number of tastants used in this study, responsive cells can be grouped by response profile (Tables C and D). Similar observations of different response profiles among bitter taste receptor cells from the oral cavity have also been reported⁴². A recent molecular study has revealed that each taste receptor cell expresses a different subset of receptor genes and varies in gene expression level⁵⁹. Similarly, differential response profiles of haploid male germ cells can be explained by the heterogeneity of Tas2r expression patterns. In fact, our in situ hybridization results and transgenic studies (FIG. 2 and data not shown) show that many but not all spermatogenic cells express Tas2r105. This supports the conclusion of heterogeneous expression patterns of Tas2rs in male germ cells. The variations in response intensity to the same stimuli may be partially due to the activation of multiple receptors by the same compound. Cells expressing the cognate receptors may have stronger responses than cells carrying non-cognate receptors. Comparative analysis of response profiles of testicular spermatids versus epididymal sperm cells identifies contrasting changes in the profiles (Tables C and D). Although the molecular and cellular events for this shift in responsiveness are yet to be defined, we hypothesize that after Tas2r genes are transcribed during the meiotic phase, only a few receptor proteins along with downstream signaling components are sorted to the cell surface and accessible to microenvironment stimuli. During the maturation process from testicular spermatids to epididymal sperm, additional receptors are localized to the cell surface, and each cell becomes responsive to more environmental ligands.

We believe that Tas2rs expressed in mammalian male germ cells have functions similar to those of Tas2rs in the digestive and respiratory systems: to detect the toxic bitter-tasting compounds. A large number of naturally occurring and synthetic bitter-tasting substances have been identified, and many of them are present in foodstuffs and medicine^{24,60}. These compounds display an enormous diversity of chemical structures. Many of these bitter tastants are hydrophobic and can readily cross cell membranes^{61,62}, suggesting that they can pass the blood testis barrier as well. Other ligands of the TAS2R human receptors are listed herein and the DB website⁸².

Because of the heterogeneity of the expressed Tas2rs among the spermatogenic cells and sperm, a given bitter compound may be able to activate a subset of these cells. Because of polymorphisms in Tas2r genes, the impact of a given bitter compound on the spermatogenesis, sperm maturation, and fertilization may vary among individuals^{63,64}.

Calcium signaling pathways play critical roles from spermatogenesis to fertilization⁶⁵. Tas2r mediated increases in intracellular calcium concentrations affects many molecular and cellular steps, including those that may lead to altered DNA or histone modifications, chromatin packaging, sperm motility, and fertilization. In the oral cavity, taste bud cells utilize a heterotrimeric G-protein consisting of β-gustducin, Gβ3, and β13 subunits, and the effector enzyme phospholipase C 2 (PLCβ2) and inositol trisphosphate (IP3) receptor type 3 IP3R3 to release calcium ions from the intracellular stores, which are sequestered back into the endoplasmic reticulum by the sarco/endoplasmic reticulum Ca2+-ATPase Serca 3⁶⁶⁻²¹. Gustducin has been reported to be expressed in spermatids as well²². Testicular Tas2r receptors are theorized to utilize a similar signal transduction pathway.

Chemotaxis is also known to play an important role in fertilization⁷³. Olfactory receptors, in addition to bitter taste receptors, have been found in mammalian germ cells, and activation of these receptors by some volatile odorants attracts sperm⁷⁴⁻⁷⁷. Caffeine has been employed to induce sperm hyperactivation and improve artificial insemination success rates⁷⁸⁻⁷⁹. The inventors theorize that other bitter-tasting compounds that occur in foods may also affect sperm behavior.

All publications cited in this specification, including particularly US provisional applications No. 61/613,397, filed Mar. 20, 2012; No. 61/616,382 filed Mar. 27, 2012 and No. 61/696,577, filed Sep. 4, 2012, the sequence of any publically available nucleic acid and/or peptide sequence cited throughout the disclosure, and the Sequence Listing, are incorporated herein by reference. While the invention has been described with reference to particular embodiments, it will be appreciated that modifications can be made without departing from the spirit of the invention. Such modifications are intended to fall within the scope of the appended claims.

TABLE E
(Sequence Listing Free Text) The following information is provided for
sequences containing free text under numeric identifier <223>.

| SEQ ID NO: (con- | Free text |
|---|---|
| taining free text) | under <223> |
| 1 | Primer |
| 2 | Primer |
| 3 | Primer |
| 4 | Primer |
| 5 | Primer |
| 6 | Primer |
| 7 | Primer |
| 8 | Primer |
| 9 | Primer |
| 10 | Primer |
| 11 | Primer |
| 12 | Primer |
| 13 | Primer |
| 14 | Primer |
| 15 | Primer |
| 16 | Primer |
| 17 | Primer |
| 18 | Primer |
| 19 | Primer |
| 20 | Primer |
| 21 | Primer |
| 22 | Primer |
| 23 | Primer |
| 24 | Primer |
| 25 | Primer |
| 26 | Primer |
| 27 | Primer |
| 28 | Primer |
| 29 | Primer |
| 30 | Primer |
| 31 | Primer |
| 32 | Primer |
| 33 | Primer |
| 34 | Primer |
| 35 | Primer |
| 36 | Primer |
| 37 | Primer |
| 38 | Primer |
| 39 | Primer |
| 40 | Primer |
| 41 | Primer |
| 42 | Primer |
| 43 | Primer |
| 44 | Primer |
| 45 | Primer |
| 46 | Primer |
| 47 | Primer |
| 48 | Primer |
| 49 | Primer |
| 50 | Primer |
| 51 | Primer |
| 52 | Primer |
| 53 | Primer |
| 54 | Primer |
| 55 | Primer |
| 56 | Primer |
| 57 | Primer |
| 58 | Primer |
| 59 | Primer |
| 60 | Primer |
| 61 | Primer |
| 62 | Primer |
| 63 | Primer |
| 64 | Primer |
| 65 | Primer |
| 66 | Primer |
| 67 | Primer |
| 68 | Primer |
| 69 | Primer |
| 70 | Primer |
| 71 | Primer |
| 72 | Primer |
| 73 | Primer |
| 74 | Primer |
| 75 | Primer |
| 76 | Primer |
| 77 | Primer |
| 78 | Primer |
| 79 | Primer |
| 80 | Primer |
| 81 | Primer |
| 82 | Primer |
| 83 | Primer |
| 84 | Primer |
| 85 | Primer |
| 86 | Primer |
| 87 | Primer |
| 88 | Primer |
| 89 | Primer |
| 90 | Primer |

### REFERENCES

1. Eddy, E. M. (2002) Recent Prog Horm Res 57, 103-128
2. Ronquist, G., and Brody, I. (1985) Biochim Biophys Acta 822, 203-218
3. Park, K. H et al., Sci Signal 4, ra31
4. Suarez, S. S. (2008) Hum Reprod Update 14, 647-657
5. Iguchi, N et al., (2006) Proc Natl Acad Sci USA 103, 7712-7717
6. Almstrup, K., et al, (2004) Biol Reprod 70, 1751-1761
7. Schlecht, U et al., (2004) Mol Biol Cell 15, 1031-1043
8. Schultz, N et al., (2003) Proc Natl Acad Sci USA 100, 12201-12206
9. Shima, J. E., et al (2004) Biol Reprod 71, 319-330
10. Wu, S. M., et al (2004) Genomics 84, 971-981
11. Hong, S., et al (2005) J Biol Chem 280, 7685-7693
12. de Rooij, D. G., and de Boer, P. (2003) Cytogenet Genome Res 103, 267-276
13. Matzuk, M. M., and Lamb, D. J. (2002) Nat Cell Biol 4 Suppl, s41-49
14. Matzuk, M. M., and Lamb, D. J. (2008) Nat Med 14, 1197-1213
15. Hew, K. W., et al (1993) Toxicol Appl Pharmacol 121, 15-21
16. Feist, G. W., et al (2005) Environ Health Perspect 113, 1675-1682
17. Cederroth, C. R., et al (2010) Int J Androl 33, 304-316
18. Howard, T. D., et al, PLoS One 6, e26791
19. Adler, E., et al (2000) Cell 100, 693-702
20. Matsunami, H et al., (2000) Nature 404, 601-604
21. Chandrashekar, J., et al (2000) Cell 100, 703-711
22. Glendinning, J. I. (1994) Physiol Behav 56, 1217-1227
23. Dubois, G. E., et al (2008) Chemistry of Gustatory Stimuli. In Olfaction and taste (Firestein, S., and Beauchamp, G.K. ed.), Elsivier, Amsterdam. pp 48
24. Maehashi, K., and Huang, L. (2009) Cell Mol Life Sci 66, 1661-1671
25. Hofmann, T. (2009) Ann N Y Acad Sci 1170, 116-125
26. Wu, S. V et al (2002) Proc Natl Acad Sci USA 99, 2392-2397
27. Rozengurt, N., et al (2006) Am J Physiol Gastrointest Liver Physiol 291, G792-802
28. Finger, T. E., et al (2003) Proc Natl Acad Sci USA 100, 8981-8986
29. Sbarbati, A., et al, (2004) J Comp Neurol 475, 188-201
30. Tizzano, M., et al, (2010) Proc Natl Acad Sci USA 107, 3210-3215
31. Tizzano, M., et al, (2011) BMC Pulm Med 11, 3
32. Shah, A. S. et al, (2009) Science 325, 1131-1134
33. Deshpande, D. A. et al, (2010) Nat Med 16, 1299-1304
34. Singh, N., et al., Biochem Biophys Res Commun 406, 146-151
35. Schaeren-Wiemers, N., and Gerfin-Moser, A. (1993) Histochemistry 100, 431-440
36. Braissant, O., and Wahli, W. (1998) Endocrinology 139, 2748-2754
40. Xia, J., et al (2007) Biol Reprod 77, 551-559
41. Gomez, G. et al, (2005) Chem Senses 30, 317-326
42. Caicedo, A., and Roper, S. D. (2001) Science 291, 1557-1560
43. Sharpe, R. M. (2010) Philos Trans R Soc Lond B Biol Sci 365, 1697-1712
44. Amory, J. K. (2007) Drugs Today (Barc) 43, 717-724
45. Huynh, P. N., et al, (2000) J Androl 21, 689-699
46. Anway, M. D. et al (2005) Science 308, 1466-1469
47. Carone, B. R., et al, (2010) Cell 143, 1084-1096
48. Howard, T. D. et al, (2011) PLoS One 6, e26791
49. Bachmanov, A. A., and Beauchamp, G. K. (2007) Annu Rev Nutr 27, 389-414
50. Wu, S. V., Chen, M. C., and Rozengurt, E. (2005) Physiol Genomics
51. Kierszenbaum, A. L., and Tres, L. L. (1978) Fed Proc 37, 2512-2516
52. Behrens, M. et al (2004) Biochem Biophys Res Commun 319, 479-485
53. Meyerhof, W. et al, (2010) Chem Senses 35, 157-170
54. Bufe, B. et al, (2005) Curr Biol 15, 322-327
55. Kim, U. K., et al (2003) Science 299, 1221-1225
56. Ren, D., and Xia, J. (2010) Physiology (Bethesda) 25, 165-175
57. Park, K. H. et al, (2011) Sci Signal 4, ra31
58. Visconti, P. E. (2009) Proc Natl Acad Sci USA 106, 667-668
59. Behrens, M., et al, (2007) J Neurosci 27, 12630-12640
60. Belitz H D, W. H. (1985) Food Reviews International. 1, 271-354
61. Peri, I., et al, (2000) Am J Physiol Cell Physiol 278, C17-25
62. Kumazawa T, N. T., Kurihara K. (1988) Biochemistry 27, 1239-1244
63. Delwiche, J. F. et al, (2001) Percept Psychophys 63, 761-776
64. Anonymous. (1931) Science 73 (suppl), 14
65. Publicover, S., et al (2007) Nat Cell Biol 9, 235-242
66. Wong, G. T., et al (1996) Nature 381, 796-800
67. Huang, L., et al (1999) Nat Neurosci 2, 1055-1062
68. Rossler, P. et al (1998) Eur J Cell Biol 77, 253-261
69. Clapp, T. R. et al, (2001) BMC Neurosci 2, 6
70. Hisatsune, C. et al, (2007) JBiol Chem 282, 37225-37231
71. Iguchi, N. et al (2011) PLoS One 6, e2316
72. Fehr, J. et al, (2007) J Comp Physiol A Neuroethol Sens Neural Behav Physiol 193, 21-34
73. Garbers, D. L. (1989) Annu Rev Biochem 58, 719-742
74. Veitinger, T. et al, (2011) JBiol Chem 286, 17311-17325
75. Vanderhaeghen, P. et al, (1993) J Cell Biol 123, 1441-1452
76. Spehr, M. et al, (2003) Science 299, 2054-2058
77. Parmentier, M., et al. (1992) Nature 355, 453-455
78. Yamaguchi, S. et al, (2009) J Reprod Dev 55, 645-649
79. Colas, C. et al, (2009) Int J Androl 33, e187-197
80. Meyer, D. et al, (2012) PloS One, 7(2):e32354
81. International Patent Publication No. WO2006/133691
82. Bitter D B website, of the Institute of Biochemistry, Food Science and Nutrition, Faculty of Agriculture, The Hebrew University of Jerusalem (http://bitterdb.agri.huji.ac.il./bitterdb/).
83. Bhal et al, 2007 Mol. Pharmaceutics, 4(4):556-560
84. Sambrook, et al 3^{rd} Edit. Molecular Cloning, A Laboratory Manual., Cold Spring Harbor Laboratory, Cold Spring Harbor, N.Y. 2001.
85. P. J. Goodford, (1985) J. Med. Chem., 1985 28:849-857
86. A. Miranker and M. Karplus, (1991) Proteins: Structure, Function and Genetics, 11:29-34
87. D. S. Goodsell and A. J. Olsen, (1990) Proteins: Structure, Function, and Genetics, 8:195-202)
88. I. D. Kuntz et al. (1982)J. Mol. Biol., 1982 161:269-288
89. Rusinko, A., (1993) Chem. Des. Auto. News, 8:44-47
90. P. A. Bartlett et al. (1989) “CAVEAT: A Program to Facilitate the Structure-Derived Design of Biologically Active Molecules”, in Molecular Recognition in Chemical and Biological Problems”, Special Pub., Royal Chem. Soc. 78, pp. —182-196 (1989)], which is available from the University of California, Berkeley, Calif.
91. Y. C. Martin, (1992) J. Med. Chem., 35:2145-2154)
92. H.-J. Bohm, (1992) J. Comp. Aid. Molec. Design, 6:61-78
93. Y. Nishibata and A. Itai, (1991) Tetrahedron, 47:8985
94. “Handbook of Pharmaceutical Excipients”, 5^{th} Edition, Eds.: Rowe, Sheskey, and Owen, APhA Publications (Washington, D.C.), Dec. 14, 2005
95. U.S. Pat. No. 7,078,053
96. N. C. Cohen et al. (1990) J. Med. Chem., 33:883-894.
97. M. A. Navia and M. A. Murcko, (1992) Current Opinions in Structural Biology, 2:202-210.
98. P. S. Farmer, Drug Design, Ariens, E. J., ed., Vol. 10, pp 119-143 (Academic Press, New York, 1980);
99. U.S. Pat. No. 5,331,573
100. U.S. Pat. No. 5,500,807
101. C. Verlinde, (1994) Structure, 2:577-587
102. I. D. Kuntz, (1992) Science, 257:1078-1082.
103. US Patent Application Publication No. 2011/0014634
104. U.S. Pat. No. 7,723,481
105. U.S. Pat. No. 7,105,650
106. U.S. Pat. No. 8,030,451
107. U.S. Pat. No. 8,030,009
108. U.S. Pat. No. 8,080,008
109. US Patent Application Publication No. 2007/0077545

1. A composition for modifying fertility in a mammalian subject comprising a ligand that binds, inhibits or activates a TAS2R receptor in a pharmaceutically acceptable carrier.

2. The composition according to claim 1, wherein said modifying comprises enhancing fertility or decreasing or preventing fertility.

3. (canceled)

4. The composition according to claim 1, wherein said composition is in the form of:
(a) a topical cream, ointment, or transdermal patch for topical delivery of the ligand,
(b) an implant for timed release in vivo of the ligand,
(c) a condom or other contraceptive implant or device in which said ligand is present or on which said ligand is coated,
(d) a timed release formulation,
(e) a composition for collecting, storing or transporting or administering sperm for insemination or IVF procedures, or
(f) a rinse, douche or vaginal treatment for use in a female prior to fertilization.

5. (canceled)

6. The composition according to claim 1, for in vivo administration to a male subject, wherein said ligand is present in an amount sufficient to increase or decrease the quality and/or quantity of sperm produced by said subject.

7-9. (canceled)

10. The composition according to claim 1, for intravaginal contraceptive administration to a female subject, wherein said ligand is present in an amount sufficient to decrease the quality and/or quantity of sperm with which it comes in contact.

11. The composition according to claim 1, comprising other known contraceptive agents.

12. A method of modifying fertility in a mammalian subject comprising
(a) contacting a male subject's testis cells, germ cells or sperm with a sufficient amount of a composition comprising a ligand that binds, activates, or inhibits activation of, a TAS2R receptor expressed on the cells, or
(b) contacting male sperm present in a female subject's reproductive system prior to fertilization with a sufficient amount of a ligand that inhibits or activates, TAS2R receptors expressed on the sperm.

13. The method according to claim 12, wherein
(c) the contacting of step (a) occurs in vivo and comprises administering the ligand to a male subject, or
(d) the contacting step of (c) comprises administering the composition topically, via a crème or patch, by intravenous injection, or via an implant, or
(e) the contacting of step (a) occurs ex vivo and comprises treating a sample of a mammalian subject's sperm with a sufficient amount of the ligand, or
(f) the contacting of step (e) occurs during collection, treatment, storage, transportation, or administration of sperm as part of an in vitro fertilization (IVF) procedure.

14. (canceled)

15. The method according to claim 12, wherein the mammalian TAS2R receptor is a human TAS2R receptor, or a homolog or ortholog thereof from a non-human mammal.

16. The method according to claim 15, wherein the TAS2R receptor is a human receptor selected from TAS2R1, TAS2R3, TAS2R4, TAS2R5, TAS2R7, TAS2R8, TAS2R9, TAS2R10, TAS2R13, TAS2R14, TAS2R16, TAS2R38, TAS2R39, TAS2R40, TAS2R41, TAS2R42, TAS2R43, TAS2R44, TAS2R45, TAS2R46, TAS2R47, TAS2R48, TAS2R49, TAS2R50, or TAS2R60.

17. The method according to claim 12, wherein the ligand is selected from: caffeine, N-phenylthiourea (PTC), 6-propyl-2 thiouracil (PROP), picrotin, salicin, lupulon, lupulone, humulon, humulone, arborescin, cascarillin, parthenolide, diphenidol, diphenylthiourea, sulfocarbanilide, sym-diphenylthiourea, thiocarbanilide, thiamine, chloramphenicol, yohimbine, amarogentin, adhumulone, cohumulone, colupulone, isoxanthohumol, xanthohumol, dextromethorphan sodium, thiocyanate, trans-isohumulone, trans-isocohumulone, adlupulone, cis-isocohumulone, cis-isoloadhumulone, trans-isoadhumulone, sodium cyclamate, picrotoxinin, chloroquine, quinine, artemorin, campher, parthenolide, quassin, azathioprine, chlorpheniramine, brucine, colchicine, dapsone, denatonium benzoate, 1,10-phenanthroline, absinthin, papaverine, cromolyn, ofloxacin, procainamide, pirenzapin, benzoin, arglabin, coumarin, cucurbitacin B, haloperidol, thujone, cucurbitacin E, cucurbitacins, cycloheximid, cycloheximide, erythromycin, strychnine, famotidine, aristolochic acid, falcarindiol, noscapine, benzamide, carisoprodol, chlorhexidine, diphenhydramine, divinyl sulfoxide, flufenamic acid, sodium benzoate, trans-, 8-prenylnaringenin, amygdalin D, arbutin, helicon, D-salicin, sinigrin, phenyl beta-D-glucopyranoside, sodium thiocyanate, acetylthiourea, allyl isothiocyanate, caprolactam, dimethylthioformamide, ethylpyrazine, N-ethylthiourea, ethylene thiourea, N,N-ethylene thiourea, limonin, methimazole, 6-methyl-2-thiouracil, N-methylthiourea, phenethyl, isothiocyanate, phenylthiocarbamide (PTC), propylthiouracil, acetaminophen, acesulfame K, aloin, grossheimin, saccharin, andrographolide, cnicin, crispolide, hydrocortisone, orphenadrine, tatridin B, or andrographolide.

18. The method according to claim 12, wherein said mammalian subject is a human, a domestic animal, a livestock animal, a laboratory animal or a pest animal.

19. The method according to claim 12, wherein the contacting of the ligand with the TAS2R receptor in step (a) increases the quality and/or quantity of sperm produced by said subject, or wherein the contacting of the ligand with the TAS2R receptor in step (a) decreases the quality and/or quantity of sperm produced by said male subject.

20-21. (canceled)

22. The method according to claim 12, wherein the composition dosage is from about 1 nM to 10 mM ligand.

23. (canceled)

24. The method according to claim 12, step (b), wherein the ligand is present in a douche or vaginal treatment administered prior to or during insemination or IVF.

25. The method according to claim 15, wherein the homolog is a receptor having an amino acid sequence that is at least 30% to at least 99% homologous to a human TAS2R receptor.

26. (canceled)

27. The method according to claim 12, step (b), wherein the ligand is administered prior to or during insemination or IVF treatment to enhance the chances of conception, or wherein the ligand is administered intravaginally prior to fertilization, or wherein the ligand is present in a composition used as a douche, vaginal treatment, or contraceptive treatment or device to reduce fertility.

28-29. (canceled)

30. A method for screening a test molecule for its effect on fertility comprising:
(a) contacting a mammalian sperm cell, testis cell or cell line expressing a TAS2R receptor ex vivo with a test molecule; and
(b) assaying the contacted cells or cell lines for a change in a physical or functional characteristic of the contacted cell or cell line in comparison with a reference cell or cell line contacted with a control molecule;
wherein a change in the physical or functional characteristic of the test molecule contacted cells or cell lines vs. the reference indicates a modifying effect of the test molecule on the quality, quantity or viability of the sperm.

31. The method according to claim 30, wherein the assaying step comprises:
i. assaying the contacted cell or cell line for a change in the cytosolic calcium concentration indicative of TAS2R receptor activation in comparison with a reference cell or cell line contacted with a control molecule; wherein an increase or decrease in cytosolic calcium concentration of the test cell or cell line vs. reference indicates a modifying effect if the test molecule on the quality, quantity or viability of sperm, or
ii. a high-throughput method comprising multiple cells, cell lines, test molecules and references, or
iii. an imaging assay.

32-33. (canceled)
